SEQUENCE LISTING (39 sequences)

SEQ ID NO: 1 - protein, 828 aa, Yarrowia lipolytica
SPDWYPTPEIGSITSDWADALGESMDILAQLTLPEKVNITTGTGWRGGQCVGNTGAVPRLGIKGLCLQDGPLGVRFADFVNVFPCQNAMAATFDRILVHQRGTAIGRQSRLKGVDVHLGPVVGPLGRHATGGRNWEGFSPDPYLSGKLAFEAILGVQEEGVLATIKHFIGNEQEHYRRAEEWRDGFGFKDLKDAVSSNIEDRALHELYMWPFADAVRANVGSVMCSYNYVNGTQACQNSDLLNGKLKSELGFQGFVMSDWFAQGSGVSNALAGMDMSMPGNDVDELETVFWGEQLTRMVANGTLPEARLDDMVLRILTPLIYFGIDDRTPNFSSFVDTTVGSPHPAAKHSKKVKDVITNYHLDVRDQFAANVALDSARGAVVLLFNDGILPLKNISAIGVFGVGSRLGPNGAVCGENMQCSDGALIEGWGSGTAYPTEYESPYEALHKKASLLEVSVTGTTESWDMRLPLELAGDTDVNIVYVLANSGESTANVDKNLGDRRNVSLWHNGDELINTVASQGQTVVVVTTVGQVDMTAWLNHPNISAVLLTAPAGDYGGKAIADVLFGEVNPSGKLPYTIAANTSDYIPIVTKIPRDGAPQSDFVEGIYLDYKWYDKFERTPLYEFGYGLSYTTYSFSNLHLDVKEISEFLPPRPVPVQVTKPKMTNIDIEDLYVPNDFKMIDGLVYPWILNASAPLADSQTQFPFANGAGHVSDASGGVGGHPWLWSNAVTVTHNTTNCGDVAGRVVSQLYVAFPETLIDSPPVQLRGFDKSKLLNPGESQMTEYNLNWRDLAIWDVELQSWRVQRGEYSVYIGHSSREFELCETFTL

SEQ ID NO: 2 - protein, 852 aa, Yarrowia lipolytica
DPFSDKDAYKHSPPYYPAPEIGRVPTDLRWRAALKVAQGMVANMTLLEKVNITTGTGWEMGPCVGNTGTVERLGIKSLCLQDGPLGIRFADLITTFPAGITIASTFSRQLVRERGAAMGRENRRKGVDITLSPVVGPLGRHANGGRIWEGFSADPYLAGKLAAEAVTGIQGQNVMAVVKHMVGNEQEHFRQLGEWQGFGFKDLKQPLSSNIDDRTLNEAYLWPFADAVRANVGSVMCSYQQINGSQGCQNAHILNGKLKEEMGFQGFVMSDWLAQRSGVASVLAGLDMSMPGDGLVWADGVPLMGYELTRSVLNGTIDESRVDDMVTRILTPILYLSITPTDPNFSSWTNDTTSYKYYGAKAGGNVTVNRHIDVRDQYTTKAALDGANAALVLLKNEKKTLPLNPTNIGNLNIFGIGSKTGPLGAVCGENMQCSDGALIEGWGSGSVYPTDYQSPYDAIKERASKDNITIGGTTQSWGNLSNVEILSAAADASVVFVLSDSGESTGIVDGNIGDRNNLTLWHNGDEVVKAVASKNPNTIVVVTTVGPVNLEKWIDNPNVTAVLLTGPAGDFGGRAAASILFGDIAPSGKLPFTIAKNDTDYIPLTTKIPEDGLPQDYFTEGTLLDYKRFDENQVTPRFEFGYGLSYSNITVENLEARYAFPSIPEFLPTPFAPSNPNKPKNAFTPHANESVFPSDIDPLNKYVYPYLNDTSEIFSNETHYPYPEGYSSEQSNSTNINGGAVGGNPALWLSAVYIVHSVSNYGPYDTGVVTQMYIAFPQDNDDLKTAPRQLRGFERSELKVGERQGILYDVQWRDLAVWDVKLQSWRVQRGEYKVYVGHSSRDFVLTTSFTLK

SEQ ID NO: 3 - DNA, 2535 bp, Yarrowia lipolytica
  CDS (1)..(2535)
  sig_peptide (1)..(48)
atgatcttctctctgcaactactactgacgacggcactagcggcctcttcgcctgactggtaccccacacccgaaatcggttcaattaccagtgattgggccgatgctcttggtgagtccatggacatcctggcccaattgactcttcccgaaaaggtcaacatcaccaccggtaccggatggagaggtgggcagtgtgttggaaacacaggggctgttcctcgtctcggaatcaagggcctgtgtctccaagacggtcctctgggcgtccgttttgccgactttgtcaatgtcttcccttgccagaacgcaatggccgccacctttgatcgtatcctagttcaccaacgaggaaccgctattggacgtcagtctagactcaagggagtcgatgttcatctcggaccagtggtgggacctcttggacgacacgctaccggcggaagaaactgggaaggtttctcccccgacccatatctgtctggaaagctcgccttcgaagcaattcttggagtccaggaagagggagttcttgcaaccatcaagcacttcattggaaacgaacaagaacattaccggcgagccgaagagtggagagacggctttggatttaaagacctgaaggacgccgtctcttccaacattgaagacagagctctacatgagttgtacatgtggccgtttgccgatgctgttagggctaatgtcggctcagtcatgtgctcctacaactacgtgaacggaacccaggcttgccagaacagtgacttgctcaacggaaagctcaagtccgagctcggtttccagggctttgtcatgtccgactggtttgctcagggaagcggagtgtctaacgctctggctggaatggacatgagtatgcctggaaatgacgttgatgagttagaaactgtcttctggggagaacagctgacccgaatggttgccaacggtacccttccagaagcccgtctcgatgatatggttctgcgaattctgacccctctcatctactttgggatcgacgatcgaacacccaacttctcctcctttgtcgacactacagtgggaagtccccaccccgctgctaagcactccaagaaagtcaaggatgttatcaccaactaccatctcgatgtgcgagaccagtttgcagccaatgttgctcttgatagtgctcgaggagctgttgttctgcttttcaatgacggtattcttcccctgaagaacatttccgctattggagtatttggagtcggctctagacttggccccaatggagctgtttgtggggaaaacatgcagtgctcagacggagctcttatcgaaggatggggaagtggaactgcttaccctacggagtacgaaagcccttacgaagctctccacaaaaaggcttctctacttgaagtgtcagtgacaggaaccaccgagtcatgggatatgagacttcctcttgagctggctggagacactgacgtgaatattgtttatgtattggcaaattcaggagagtctactgccaacgttgataagaaccttggggatcgccgaaatgtcagtctatggcacaatggcgatgaacttattaatacagttgccagtcagggacagactgtcgttgttgtcactacggttggacaagttgacatgaccgcttggctcaaccaccccaacatcagtgccgttcttctgactgctcctgctggtgattacggaggaaaagccatcgctgatgtgttatttggagaggttaatccctcaggaaagctgccttatactatcgcagcaaatacttctgattatattcctattgtcaccaagatccctcgagatggagctccccagtccgactttgtggagggaatctatcttgactacaagtggtacgacaagtttgaaaggactcccctctacgaatttggttacggtctgtcatacaccacctactccttcagcaatctgcatcttgatgttaaggagattagtgagttccttcctccccggcctgttcctgtacaggttactaaacccaagatgaccaatatcgacattgaggacctttatgtccccaatgactttaaaatgattgacggtcttgtttacccttggattctcaacgccagtgcgcccctcgcggactctcagactcagttcccctttgcaaatggagctggacatgtcagtgacgcttctggaggagtgggtggtcacccctggctttggtctaacgctgttactgtcactcacaacaccaccaactgcggtgatgttgctggacgagtagtttctcagctgtacgttgccttccctgaaacccttatcgactctcctccggtgcagcttcgaggattcgacaagtccaagctcctgaaccctggtgagtctcagatgaccgagtacaacctcaactggcgagatctggccatttgggacgtggaactgcagagctggagagtgcagcgaggcgagtattccgtgtacattggtcattccagccgagaattcgagctatgtgagactttcactttgtag

SEQ ID NO: 4 - protein, 844 aa, Yarrowia lipolytica
MIFSLQLLLTTALAASSPDWYPTPEIGSITSDWADALGESMDILAQLTLPEKVNITTGTGWRGGQCVGNTGAVPRLGIKGLCLQDGPLGVRFADFVNVFPCQNAMAATFDRILVHQRGTAIGRQSRLKGVDVHLGPVVGPLGRHATGGRNWEGFSPDPYLSGKLAFEAILGVQEEGVLATIKHFIGNEQEHYRRAEEWRDGFGFKDLKDAVSSNIEDRALHELYMWPFADAVRANVGSVMCSYNYVNGTQACQNSDLLNGKLKSELGFQGFVMSDWFAQGSGVSNALAGMDMSMPGNDVDELETVFWGEQLTRMVANGTLPEARLDDMVLRILTPLIYFGIDDRTPNFSSFVDTTVGSPHPAAKHSKKVKDVITNYHLDVRDQFAANVALDSARGAVVLLFNDGILPLKNISAIGVFGVGSRLGPNGAVCGENMQCSDGALIEGWGSGTAYPTEYESPYEALHKKASLLEVSVTGTTESWDMRLPLELAGDTDVNIVYVLANSGESTANVDKNLGDRRNVSLWHNGDELINTVASQGQTVVVVTTVGQVDMTAWLNHPNISAVLLTAPAGDYGGKAIADVLFGEVNPSGKLPYTIAANTSDYIPIVTKIPRDGAPQSDFVEGIYLDYKWYDKFERTPLYEFGYGLSYTTYSFSNLHLDVKEISEFLPPRPVPVQVTKPKMTNIDIEDLYVPNDFKMIDGLVYPWILNASAPLADSQTQFPFANGAGHVSDASGGVGGHPWLWSNAVTVTHNTTNCGDVAGRVVSQLYVAFPETLIDSPPVQLRGFDKSKLLNPGESQMTEYNLNWRDLAIWDVELQSWRVQRGEYSVYIGHSSREFELCETFTL

SEQ ID NO: 5 - DNA, 2610 bp, Yarrowia lipolytica
  CDS (1)..(2610)
  sig_peptide (1)..(51)
atgctcgcattcgtcctactgctgacgatgctgctcgcagcagcgcttgctgacccgttctcagataaagacgcttacaaacacagccctccatactaccctgctccggagattggcagagttcccaccgacctgcgatggagagctgccttgaaggtggcccagggtatggtcgctaacatgacactgcttgagaaggtgaacatcaccaccggtacgggctgggagatgggtccttgtgttggaaacactggtaccgtcgaacgactgggtataaaatcgctgtgccttcaagacggccctcttgggattcgatttgctgacctcattaccacattccctgctggtatcactattgcctctaccttctctcgacagctggttagagagcgaggtgctgctatgggacgggagaatagacgcaagggagtggatatcactctcagccctgtggttggaccactgggaagacatgctaacggaggtcgaatctgggagggcttctctgctgacccctaccttgctggaaagctcgccgccgaggccgtgacaggtatacagggccagaacgtcatggctgtggtaaagcatatggttggaaacgagcaggaacattttcgacaacttggcgagtggcagggattcggattcaaggatctgaagcagcccctctcttcaaacatcgacgaccgaactcttaacgaagcgtacctttggccctttgctgatgctgttcgagccaatgttggatctgtcatgtgttcctatcagcagatcaatggctctcagggttgtcaaaacgcccacattctgaacggtaagctcaaggaggaaatgggtttccagggctttgtcatgtccgactggcttgcccagcgaagtggcgtggcgtctgttcttgctggtctcgatatgagcatgcctggagacggtcttgtctgggcggacggtgttccgctcatgggatacgagttgaccaggagtgtgctgaatggaaccattgatgaaagccgagtggacgacatggttacccgaatccttacccccatactgtatctctcaattactccgaccgaccccaacttcagctcttggaccaacgacactactagctacaagtactacggagccaaggctggtggaaacgtcactgttaaccgacatattgatgtgagagaccagtacactaccaaggctgctcttgatggagcaaacgctgcgcttgttcttctcaagaatgagaagaagactcttcctctgaaccctaccaatattggaaacctcaacattttcggtattggttctaaaaccggcccacttggagctgtctgtggagaaaatatgcagtgtagtgatggcgcccttattgagggatgggggtccggttccgtctaccctaccgattatcaatctccttacgacgccattaaggagagagcctccaaggacaacatcaccatcggaggcactacgcaatcttggggtaacctgtcgaacgttgagatcctttcagctgctgccgacgccagtgtcgtctttgttctttccgactccggtgagagtactggtattgttgacggcaacattggggatcgaaacaacttgacgctgtggcacaatggagacgaggttgtcaaggctgtggcatctaagaaccccaacactatcgttgttgttaccactgtcggccctgtgaacctcgaaaagtggatcgacaacccaaacgtcactgccgtgcttctcactggacccgctggtgactttggaggaagagctgcagcctctattctcttcggcgacatcgccccttcaggaaaactccctttcactattgccaagaatgacaccgactacattcctcttactactaagatccctgaagacggccttcctcaagactatttcactgagggtactcttttggactacaaacggttcgacgagaaccaggtgactcctaggtttgaatttggctacggtctgtcttactctaacattacggtggagaatctcgaagcccggtatgctttccctagcattcctgagttcttgcccactccctttgccccttcgaaccccaacaagcctaagaacgcatttactcctcacgccaatgagtccgtcttccccagtgacattgatcctttgaacaagtacgtctatccatacctgaacgatacctcggagatcttctctaacgagacccattatccctatcctgaggggtactccagtgagcagtccaacagtaccaacattaacggcggggctgtcggaggcaaccctgctctgtggctctctgcagtctacattgtccacagcgtgtctaactatggtccctatgatactggagtggtcacccagatgtacattgccttccctcaggataacgacgatcttaaaaccgctcctagacagcttcgaggattcgaacggtccgagctcaaggtgggagaacggcagggaattctatacgatgttcaatggcgagatctcgcggtctgggatgtcaaacttcagagctggcgggtccaacgaggagagtacaaggtttacgtaggccacagttcgcgagactttgttctgaccaccagcttcactctcaagtag

SEQ ID NO: 6 - protein, 869 aa, Yarrowia lipolytica
MLAFVLLLTMLLAAALADPFSDKDAYKHSPPYYPAPEIGRVPTDLRWRAALKVAQGMVANMTLLEKVNITTGTGWEMGPCVGNTGTVERLGIKSLCLQDGPLGIRFADLITTFPAGITIASTFSRQLVRERGAAMGRENRRKGVDITLSPVVGPLGRHANGGRIWEGFSADPYLAGKLAAEAVTGIQGQNVMAVVKHMVGNEQEHFRQLGEWQGFGFKDLKQPLSSNIDDRTLNEAYLWPFADAVRANVGSVMCSYQQINGSQGCQNAHILNGKLKEEMGFQGFVMSDWLAQRSGVASVLAGLDMSMPGDGLVWADGVPLMGYELTRSVLNGTIDESRVDDMVTRILTPILYLSITPTDPNFSSWTNDTTSYKYYGAKAGGNVTVNRHIDVRDQYTTKAALDGANAALVLLKNEKKTLPLNPTNIGNLNIFGIGSKTGPLGAVCGENMQCSDGALIEGWGSGSVYPTDYQSPYDAIKERASKDNITIGGTTQSWGNLSNVEILSAAADASVVFVLSDSGESTGIVDGNIGDRNNLTLWHNGDEVVKAVASKNPNTIVVVTTVGPVNLEKWIDNPNVTAVLLTGPAGDFGGRAAASILFGDIAPSGKLPFTIAKNDTDYIPLTTKIPEDGLPQDYFTEGTLLDYKRFDENQVTPRFEFGYGLSYSNITVENLEARYAFPSIPEFLPTPFAPSNPNKPKNAFTPHANESVFPSDIDPLNKYVYPYLNDTSEIFSNETHYPYPEGYSSEQSNSTNINGGAVGGNPALWLSAVYIVHSVSNYGPYDTGVVTQMYIAFPQDNDDLKTAPRQLRGFERSELKVGERQGILYDVQWRDLAVWDVKLQSWRVQRGEYKVYVGHSSRDFVLTTSFTLK

SEQ ID NO: 7 - protein, 847 aa, Yarrowia galli
  SIGNAL (1)..(16)
MIFSLQLLLTTVPVLAAYSPDWYPTPEIGTINNDWADALEQSMDILSQLTLPEKVNITTGTGWMGGQCVGNTGGVPRLGIKGLCLQDGPLGVRFADFVNVFPCQNAMAATFDRILVHQRGTAIGHQSKLKGVDVHLGPVVGPIGRHATGGRNWEGFSPDPYLSGKLAYEAIQGIQEENVLATIKHFIGNEQDHYRRAQEWRDGFNFTGLKLPVSSNNIDDRALHELYMWPFADAVKAGVGSVMCAYNNVNGTQACQNSDLLNGKLKSELGFQGFVMSDWFAQGNGVANALAGMDMSMPGTDVDEFETVFWGEQLTRMVANGTLPESRLDDMVLRILTPLMYFGIDDREPNFASFVDTTVGSPYPAAKNPKQVQDKIVNYHLDVRNQFSANVALESARGAIVLLFNSGVLPLKDKSNIGVFGVGSVIGPNGAICENMQCSDGALIEGWGSGTAYPTEYESPYEALHKKASLLDVHVTGTSESWDMSLPIEIAGSTDINIVYVLAASGESTASVDGNIGDRNNVSLWHNGDELINAVADQGETVVVVTTVGQVDMSEWLFHPNVSAVLLTAPAGDYGGKAMADVLFGEVNPSGKLPYTLADDISEYIPIVTEIPADGAPQSDFVEGIYLDYKWYDKFEKTPLYEFGYGLSYTNYTFTDLSLDIKEIDEFLPARPDPVVVTKPEDFPIDLADLYVPDGFEMIDGLVYPWIQNVSVPLSDSEAQFPFADGAGHISDASGGVGGHPWLWSVAATITHNTTNCGDVAGRAVSQLYIAFPETCVDVPLVQLRGFDKSGMLNPGESQETVYNLHWRDLAIWDVELQSWRVQRGEYAVFIGHSSRDFELFDSFTLE

SEQ ID NO: 8 - protein, 831 aa, Yarrowia galli
AYSPDWYPTPEIGTINNDWADALEQSMDILSQLTLPEKVNITTGTGWMGGQCVGNTGGVPRLGIKGLCLQDGPLGVRFADFVNVFPCQNAMAATFDRILVHQRGTAIGHQSKLKGVDVHLGPVVGPIGRHATGGRNWEGFSPDPYLSGKLAYEAIQGIQEENVLATIKHFIGNEQDHYRRAQEWRDGFNFTGLKLPVSSNNIDDRALHELYMWPFADAVKAGVGSVMCAYNNVNGTQACQNSDLLNGKLKSELGFQGFVMSDWFAQGNGVANALAGMDMSMPGTDVDEFETVFWGEQLTRMVANGTLPESRLDDMVLRILTPLMYFGIDDREPNFASFVDTTVGSPYPAAKNPKQVQDKIVNYHLDVRNQFSANVALESARGAIVLLFNSGVLPLKDKSNIGVFGVGSVIGPNGAICENMQCSDGALIEGWGSGTAYPTEYESPYEALHKKASLLDVHVTGTSESWDMSLPIEIAGSTDINIVYVLAASGESTASVDGNIGDRNNVSLWHNGDELINAVADQGETVVVVTTVGQVDMSEWLFHPNVSAVLLTAPAGDYGGKAMADVLFGEVNPSGKLPYTLADDISEYIPIVTEIPADGAPQSDFVEGIYLDYKWYDKFEKTPLYEFGYGLSYTNYTFTDLSLDIKEIDEFLPARPDPVVVTKPEDFPIDLADLYVPDGFEMIDGLVYPWIQNVSVPLSDSEAQFPFADGAGHISDASGGVGGHPWLWSVAATITHNTTNCGDVAGRAVSQLYIAFPETCVDVPLVQLRGFDKSGMLNPGESQETVYNLHWRDLAIWDVELQSWRVQRGEYAVFIGHSSRDFELFDSFTLE

SEQ ID NO: 9 - protein, 854 aa, Artificial Sequence
  BGL2 consensus sequence
  VARIANT (1)..(1): Replace = Phe
  VARIANT (5)..(5): Replace = Ala
  VARIANT (7)..(7): Replace = Arg
  VARIANT (8)..(8): Replace = Ala
  VARIANT (9)..(9): Replace = Ser
  VARIANT (25)..(25): Replace = Ile
  VARIANT (26)..(26): Replace = Leu
  VARIANT (29)..(29): Replace = Arg
  VARIANT (32)..(32): Replace = Ala
  VARIANT (33)..(33): Replace = Thr
  VARIANT (35)..(35): Replace = Met
  VARIANT (36)..(36): Replace = Glu or Asn
  VARIANT (37)..(37): Replace = Ala
  VARIANT (40)..(40): Replace = Gly, Asn or Ser
  VARIANT (41)..(41): Replace = Leu
  VARIANT (48)..(48): Replace = Leu or Tyr
  VARIANT (55)..(55): Replace = Ser
  VARIANT (69)..(69): Replace = Ser
  VARIANT (70)..(70): Replace = Pro
  VARIANT (79)..(79): Replace = Ile
  VARIANT (88)..(88): Replace = Val
  VARIANT (90)..(90): Replace = Leu
  VARIANT (91)..(91): Replace = Thr
  VARIANT (93)..(93): Replace = Lys
  VARIANT (96)..(96): Replace = Ala
  VARIANT (103)..(103): Replace = Met
  VARIANT (109)..(109): Replace = Arg
  VARIANT (110)..(110): Replace = Lys
  VARIANT (113)..(113): Replace = Gln
  VARIANT (117)..(117): Replace = Ala
  VARIANT (142)..(142): Replace = Asn
  VARIANT (145)..(145): Replace = Gly
  VARIANT (165)..(165): Replace = Gln
  VARIANT (168)..(168): Replace = Glu
  VARIANT (172)..(172): Replace = Gly
  VARIANT (182)..(182): Replace = Met or Leu
  VARIANT (199)..(199): Replace = Tyr
  VARIANT (201)..(201): Replace = Tyr
  VARIANT (217)..(217): Replace = Met
  VARIANT (229)..(229): Replace = Ile
  VARIANT (245)..(245): Replace = Ser
  VARIANT (252)..(252): Replace = Ala
  VARIANT (253)..(253): Replace = Phe or Ala
  VARIANT (254)..(254): Replace = Leu
  VARIANT (259)..(259): Replace = Ile
  VARIANT (263)..(263): Replace = Phe
  VARIANT (269)..(269): Replace = Ile
  VARIANT (299)..(299): Replace = Gly
  VARIANT (300)..(300): Replace = Leu
  VARIANT (302)..(302): Replace = Ile
  VARIANT (311)..(311): Replace = Arg
  VARIANT (312)..(312): Replace = Met
  VARIANT (318)..(318): Replace = Val
  VARIANT (319)..(319): Replace = Glu
  VARIANT (320)..(320): Replace = Glu or Gln
  VARIANT (321)..(321): Replace = Ser
  VARIANT (334)..(334): Replace = Ile
  VARIANT (335)..(335): Replace = Leu
  VARIANT (339)..(339): Replace = Leu
  VARIANT (342)..(342): Replace = Thr or Arg
  VARIANT (343)..(343): Replace = Asp or Thr
  VARIANT (352)..(352): Replace = Glu
  VARIANT (354)..(354): Replace = Tyr
  VARIANT (355)..(355): Replace = Gly
  VARIANT (356)..(356): Replace = Leu
  VARIANT (357)..(357): Replace = Glu or Gln
  VARIANT (358)..(358): Replace = Tyr or Phe
  VARIANT (359)..(359): Replace = Pro
  VARIANT (361)..(361): Replace = Asn
  VARIANT (362)..(362): Replace = Asp
  VARIANT (363)..(363): Replace = Gly or Arg
  VARIANT (365)..(365): Replace = Arg or Pro
  VARIANT (367)..(367): Replace = Ile
  VARIANT (369)..(369): Replace = Ile
  VARIANT (371)..(371): Replace = Gln
  VARIANT (373)..(373): Replace = Val
  VARIANT (374)..(374): Replace = Glu
  VARIANT (378)..(378): Replace = Gln, Ala or Ser
  VARIANT (379)..(379): Replace = Tyr
  VARIANT (381)..(381): Replace = Ile
  VARIANT (382)..(382): Replace = Gln
  VARIANT (385)..(385): Replace = Gln
  VARIANT (389)..(389): Replace = Thr
  VARIANT (398)..(398): Replace = Glu
  VARIANT (399)..(399): Replace = Gln
  VARIANT (400)..(400): Replace = Lys or Ser
  VARIANT (401)..(401): Replace = Thr
  VARIANT (405)..(405): Replace = Asn or Asp
  VARIANT (406)..(406): Replace = nothing
  VARIANT (407)..(407): Replace = Ala or nothing
  VARIANT (408)..(408): Replace = Lys
  VARIANT (409)..(409): Replace = Leu or Val
  VARIANT (410)..(410): Replace = Thr
  VARIANT (411)..(411): Replace = Thr or Lys
  VARIANT (412)..(412): Replace = Ile
  VARIANT (417)..(417): Replace = Val
  VARIANT (420)..(420): Replace = Lys or Thr
  VARIANT (423)..(423): Replace = Asn
  VARIANT (425)..(425): Replace = Ser
  VARIANT (426)..(426): Replace = nothing
  VARIANT (427)..(427): Replace = nothing
  VARIANT (430)..(430): Replace = Gln
  VARIANT (433)..(433): Replace = Glu
  VARIANT (436)..(436): Replace = Ser
  VARIANT (441)..(441): Replace = Gln or Leu
  VARIANT (451)..(451): Replace = Ser
  VARIANT (454)..(454): Replace = Leu
  VARIANT (458)..(458): Replace = Asp or Asn
  VARIANT (465)..(465): Replace = Ser or Glu
  VARIANT (466)..(466): Replace = Lys
  VARIANT (467)..(467): Replace = Asn
  VARIANT (468)..(468): Replace = Lys
  VARIANT (470)..(470): Replace = Gln
  VARIANT (471)..(471): Replace = Ile
  VARIANT (476)..(476): Replace = His or Glu
  VARIANT (482)..(482): Replace = Thr
  VARIANT (483)..(483): Replace = Ile or Arg
  VARIANT (486)..(486): Replace = Lys or Gln
  VARIANT (488)..(488): Replace = Ala
  VARIANT (490)..(490): Replace = Glu
  VARIANT (491)..(491): Replace = Ser
  VARIANT (495)..(495): Replace = Ile
  VARIANT (508)..(508): Replace = Pro
  VARIANT (513)..(513): Replace = Tyr
  VARIANT (527)..(527): Replace = Glu or Gln
  VARIANT (530)..(530): Replace = Arg
  VARIANT (531)..(531): Replace = Thr
  VARIANT (535)..(535): Replace = Lys or Gln
  VARIANT (536)..(536): Replace = Asn
  VARIANT (537)..(537): Replace = Ala
  VARIANT (556)..(556): Replace = Asp
  VARIANT (557)..(557): Replace = His
  VARIANT (575)..(575): Replace = Ser or Lys
  VARIANT (580)..(580): Replace = Val
  VARIANT (585)..(585): Replace = Val
  VARIANT (597)..(597): Replace = Arg
  VARIANT (599)..(599): Replace = Asn
  VARIANT (603)..(603): Replace = Val
  VARIANT (606)..(606): Replace = Val
  VARIANT (609)..(609): Replace = Ile
  VARIANT (611)..(611): Replace = Glu or Ser
  VARIANT (618)..(618): Replace = Phe
  VARIANT (632)..(632): Replace = Ala
  VARIANT (633)..(633): Replace = Leu
  VARIANT (634)..(634): Replace = Gln
  VARIANT (635)..(635): Replace = Lys
  VARIANT (652)..(652): Replace = Val or Leu
  VARIANT (653)..(653): Replace = Gly
  VARIANT (656)..(656): Replace = Asp or Gln
  VARIANT (657)..(657): Replace = Val
  VARIANT (658)..(658): Replace = Leu or Lys
  VARIANT (659)..(659): Replace = Tyr
  VARIANT (662)..(662): Replace = Asn
  VARIANT (663)..(663): Replace = Ser
  VARIANT (665)..(665): Replace = Ser
  VARIANT (670)..(670): Replace = Ala
  VARIANT (672)..(672): Replace = Ser
  VARIANT (673)..(673): Replace = Pro
  VARIANT (675)..(675): Replace = Ser
  VARIANT (676)..(676): Replace = Ile or Asp
  VARIANT (678)..(678): Replace = Asn
  VARIANT (681)..(681): Replace = Lys or Ala
  VARIANT (682)..(682): Replace = Asn
  VARIANT (683)..(683): Replace = Ala or Arg
  VARIANT (685)..(685): Replace = Pro
  VARIANT (686)..(686): Replace = Thr
  VARIANT (687)..(687): Replace = Hir, Ser or Glu
  VARIANT (688)..(688): Replace = Leu
  VARIANT (690)..(690): Replace = Glu or Gly
  VARIANT (692)..(692): Replace = Ile
  VARIANT (695)..(695): Replace = Ser
  VARIANT (696)..(696): Replace = Glu
  VARIANT (697)..(697): Replace = Phe
  VARIANT (698)..(698): Replace = Asp or Lys
  VARIANT (699)..(699): Replace = Gln
  VARIANT (700)..(700): Replace = Ile
  VARIANT (701)..(701): Replace = Asn, Lys or Pro
  VARIANT (702)..(702): Replace = Asn
  VARIANT (706)..(706): Replace = Thr
  VARIANT (710)..(710): Replace = Arg or Ser
  VARIANT (713)..(713): Replace = Asp
  VARIANT (714)..(714): Replace = Leu
  VARIANT (715)..(715): Replace = Ala or Asn
  VARIANT (716)..(716): Replace = nothing
  VARIANT (719)..(719): Replace = Ile
  VARIANT (720)..(720): Replace = Ser
  VARIANT (721)..(721): Replace = His or Arg
  VARIANT (728)..(728): Replace = Phe
  VARIANT (730)..(730): Replace = Ser or Val
  VARIANT (731)..(731): Replace = Glu or Val
  VARIANT (735)..(735): Replace = Ser or Gly
  VARIANT (752)..(752): Replace = Pro or Thr
  VARIANT (753)..(753): Replace = Ala
  VARIANT (754)..(754): Replace = Leu
  VARIANT (756)..(756): Replace = Val
  VARIANT (757)..(757): Replace = Ala
  VARIANT (760)..(760): Replace = Ile
  VARIANT (763)..(763): Replace = His
  VARIANT (770)..(770): Replace = Ala
  VARIANT (774)..(774): Replace = Leu
  VARIANT (777)..(777): Replace = Ser
  VARIANT (783)..(783): Replace = Glu
  VARIANT (784)..(784): Replace = Gly
  VARIANT (786)..(786): Replace = Arg or Gln
  VARIANT (801)..(801): Replace = Pro
  VARIANT (804)..(804): Replace = Asp
  VARIANT (805)..(805): Replace = Gln
  VARIANT (806)..(806): Replace = Gln or Arg
  VARIANT (808)..(808): Replace = Val
  VARIANT (809)..(809): Replace = Gln
  VARIANT (811)..(811): Replace = Asp
  VARIANT (812)..(812): Replace = Ile
  VARIANT (823)..(823): Replace = Ser
  VARIANT (824)..(824): Replace = Ile
  VARIANT (833)..(833): Replace = Lys or Asp
  VARIANT (835)..(835): Replace = Thr or Glu
  VARIANT (836)..(836): Replace = Ile
  VARIANT (837)..(837): Replace = Phe
  VARIANT (838)..(838): Replace = Ile
  VARIANT (845)..(845): Replace = Leu
  VARIANT (848)..(848): Replace = Ser or Ala
  VARIANT (850)..(850): Replace = Thr or Lys
  VARIANT (854)..(854): Replace = Lys or Asn
ADPFSDKDAYKHSPPYYPAPEIGRVPTDLRWRAALKVAQDMVANMTLIEKVNITTGTGWEMGPCVGNTGTVERLGIKSLCLQDGPLGIRFADLITTFPAGITIASTFSKQLVRERGEAMGRENRRKGVDITLSPVVGPLGRHANAGRIWEGFSADPYLAGKLAAEAVTGIQSQNVMAVVKHIVGNEQEHFRQLGEWQGFGFKDLKQPLSSNIDDRTLNEAYLWPFADAVRANVGSVMCSYQQINGSQGCQNSHILNGKLKEEMGFQGFVMSDWLAQRSGVASVLAGLDMSMPGDGLVWADGVPLMGYELTKSVLNGTIDINRVDDMVTRILTPLMYLSITPGNPNFSSWTNDTTSYKIYGAKAGGNVTVNRHIDVRDPWTTKAALDGANAALVLLKNVKNALPLKPTNIGNLNIFGIGSATGPLGAVCGENMQCSDGALIEGWGSGSVYPTDYQSPYEAIKERAAQDNITVGGTTQSWGNLSNVEILSAAADASVVFVLSDSGESTGIVDGNIGDRNNLTLWHNGDAVVKAVASRNPNTIVVVTTVGPVNLEKWINNPNVTAVLLTGPAGDFGGRAAASILFGDIAPSGKLPFTIAKNDTDYIPLTTKVPKDGLPQDYFTEGTLLDYKRFDENKVTPRFEFGYGLSYSNITMENLEARFAFPNIPEFLPTPFAPLNPHKPNDVFTPNANQSVFPDDIHPLHKYVYPYLNDTSEIFTSNETNYPYPEGYSQKQSNTTNINGGAVGGNPALWLSSVYIVHSVSNYGPYDTGVVTQMYIAFPQDNDDLKTAPRQLRGFERSELKVGERHGILYNVQWRDLAVWDVKLQSWRVQRGEYKVYVGHSSRDFVLTTSFTLQ

SEQ ID NO: 10 - protein, 874 aa, Yarrowia galli
  SIGNAL (1)..(20)
MLAFVLLLTTLQALLTAVLAADPFSDKDAYKHSPPYYPAPEIGRVPTDLRWRAALKVAQDMVANMTLIEKVNITTGTGWEMGPCVGNTGTVERLGIKSLCLQDGPLGIRFADLITTFPAGITIASTFSKQLVRERGEAMGRENRRKGVDITLSPVVGPLGRHANAGRIWEGFSADPYLAGKLAAEAVTGIQSQNVMAVVKHIVGNEQEHFRQLGEWQGFGFKDLKQPLSSNIDDRTLNEAYLWPFADAVRANVGSVMCSYQQINGSQGCQNSHILNGKLKEEMGFQGFVMSDWLAQRSGVASVLAGLDMSMPGDGLVWADGVPLMGYELTKSVLNGTIDINRVDDMVTRILTPLMYLSITPGNPNFSSWTNDTTSYKIYGAKAGGNVTVNRHIDVRDPWTTKAALDGANAALVLLKNVKNALPLKPTNIGNLNIFGIGSATGPLGAVCGENMQCSDGALIEGWGSGSVYPTDYQSPYEAIKERAAQDNITVGGTTQSWGNLSNVEILSAAADASVVFVLSDSGESTGIVDGNIGDRNNLTLWHNGDAVVKAVASRNPNTIVVVTTVGPVNLEKWINNPNVTAVLLTGPAGDFGGRAAASILFGDIAPSGKLPFTIAKNDTDYIPLTTKVPKDGLPQDYFTEGTLLDYKRFDENKVTPRFEFGYGLSYSNITMENLEARFAFPNIPEFLPTPFAPLNPHKPNDVFTPNANQSVFPDDIHPLHKYVYPYLNDTSEIFTSNETNYPYPEGYSQKQSNTTNINGGAVGGNPALWLSSVYIVHSVSNYGPYDTGVVTQMYIAFPQDNDDLKTAPRQLRGFERSELKVGERHGILYNVQWRDLAVWDVKLQSWRVQRGEYKVYVGHSSRDFVLTTSFTLQ

SEQ ID NO: 11 - protein, 854 aa, Yarrowia galli; identical to SEQ ID NO: 9

SEQ ID NO: 12 - protein, 867 aa, Yarrowia yakushimensis
  SIGNAL (1)..(17)
MLAFVLLLTSLLTAVLADPFSDKAAYKHSPPYYPAPEIGRIPTDLRWAAALEVAQNLVANMTLIEKVNITTGTGWEMGPCVGNTSPVERLGIKSLCLQDGPLGVRLTDLITAFPAGITMASTFSRQLVRERGAAMGRENRRKGVDITLSPVVGPLGRNANAGRIWEGFSADPYLAGKLAAEAVTGIQSQNVMAVVKHLVGNEQEHFRQLGEWQGYGYKDLKQPLSSNIDDRTLNEAYLWPFADAIRANVGSVMCSYQQINSSQGCQNSFILNGKIKEEMGFQGFIMSDWLAQRSGVASVLAGLDMSMPGDGLVWGLGIPLMGYELTRSVLNGTIDESRVDDMVTRILTPILYLSLTPRTPNFSSWTNDTYGYEYYGNDGGRNITINRHVDVRDAYTIQAAQDGANAALVLLKNEKNALPLDPAKLGTINIFGIGSKTGNLSCGQNMECSNGALIQGWGSGSVYPTDYLSPYNAIKERAAQNNITVGGTTHSWGNLSIVEKLSAAADASVVFVLSDSGESTGPVDGNYGDRNNLTLWHNGDQVVKAVASKNPNTIVVVTTVGPVNLEKWIDHPNVTAVLLTGPAGDFGGSAAASILFGDIAPSGKLPFTIAKNNTDYIPLVTKVPEDGLPQDYFTEGTLLDYKRFDENKVTPRFEFGYGLSYSNITVENLDVLFAFPNISEFLPAPSAPLIPHKPKNRFTPSLNESIFPSGFKQLKNYVYTYLNRTSDIASNITHYPYPEGYSSVQSNTTNINGGAVGGNPALWLPALYVVHSVSNYGPYDTGAVTQLYISFPQDNEDLRTAPRQLRGFERSELKVGDQQGVLYDIQWRDLAVWDVKLQSWRVQRGKYTVYIGHSSRDLVLSTTFTLK

SEQ ID NO: 13 - protein, 850 aa, Yarrowia yakushimensis
DPFSDKAAYKHSPPYYPAPEIGRIPTDLRWAAALEVAQNLVANMTLIEKVNITTGTGWEMGPCVGNTSPVERLGIKSLCLQDGPLGVRLTDLITAFPAGITMASTFSRQLVRERGAAMGRENRRKGVDITLSPVVGPLGRNANAGRIWEGFSADPYLAGKLAAEAVTGIQSQNVMAVVKHLVGNEQEHFRQLGEWQGYGYKDLKQPLSSNIDDRTLNEAYLWPFADAIRANVGSVMCSYQQINSSQGCQNSFILNGKIKEEMGFQGFIMSDWLAQRSGVASVLAGLDMSMPGDGLVWGLGIPLMGYELTRSVLNGTIDESRVDDMVTRILTPILYLSLTPRTPNFSSWTNDTYGYEYYGNDGGRNITINRHVDVRDAYTIQAAQDGANAALVLLKNEKNALPLDPAKLGTINIFGIGSKTGNLSCGQNMECSNGALIQGWGSGSVYPTDYLSPYNAIKERAAQNNITVGGTTHSWGNLSIVEKLSAAADASVVFVLSDSGESTGPVDGNYGDRNNLTLWHNGDQVVKAVASKNPNTIVVVTTVGPVNLEKWIDHPNVTAVLLTGPAGDFGGSAAASILFGDIAPSGKLPFTIAKNNTDYIPLVTKVPEDGLPQDYFTEGTLLDYKRFDENKVTPRFEFGYGLSYSNITVENLDVLFAFPNISEFLPAPSAPLIPHKPKNRFTPSLNESIFPSGFKQLKNYVYTYLNRTSDIASNITHYPYPEGYSSVQSNTTNINGGAVGGNPALWLPALYVVHSVSNYGPYDTGAVTQLYISFPQDNEDLRTAPRQLRGFERSELKVGDQQGVLYDIQWRDLAVWDVKLQSWRVQRGKYTVYIGHSSRDLVLSTTFTLK

SEQ ID NO: 14 - protein, 866 aa, Yarrowia alimentaria
  SIGNAL (1)..(16)
MFSLLLLLTLLSTALADPFADRDSYKHSPPYYPAPEIGRVLTDRRWRTAMNAAQSLVANMTLYEKVNITSGTGWEMGPCVGNTGTVERLGIKSICLQDGPLGIRFADKITAFPAGITIASTFSRKLVQERGAAMGRENRRKGVDITLSPVVGPLGRHANGGRIWEGFSADPYLAGKLAAQAVEGIQSQNVMAVVKHMVGNEQEHFRQLGEWQGFGFKDLKQPLSSNIDDRTMNEAYLWPFADAVRANVGSVMCSYQQINGSQGCQNSALLNGKLKEEFGFQGFVMSDWLAQRSGVASVLAGLDMSMPGDGLVWADGIPLMGYELTKMVLNGTVEQSRVDDMVTRILTPLLYLSITPGNPNFSSWTNETTGLQFPGADRGPNITVNQHVEVRDSYTTKAALDGATAALVLLKNVQSALPLKNVTKLNIFGVGSTTGPLGAVCGENMQCSDGALILGWGSGSVYPSDYQSPYEAIKERAEQDKIQVGGTTESWGNLTRVEQLAAESDASIVFVLSDSGESTGIVDGNIGDRNNLTLWHNGDEVVRTVASQSANTIVVVTTVGPVNLEKWINHPNVTAVLLTGPAGDFGGKAAASVLFGDVAPSGKLPFTIARNDTDYVPLTTKVPSDGLPQDFFTEGTLLDYKRFDALKKTPRFEFGYGLSYSNITLGNLQAKFAFNNIPEFLPTPFPPLDPHKPANVFPTELNGSVFPSDIDPIPKYVYPYLNSTSELNSNISRYPYPEGFSVVQSNGTNINGGAVGGNPALWLTALYVAHSISNHGPYDTGVVTQLYIAFPQDNEGLQTAPRQLRGFERSELPVGEQRGVQYDIQWRDLAVWDVSIQSWRVQRGDYEIFVGHSSRDLVLATKFTLN

SEQ ID NO: 15 - protein, 850 aa, Yarrowia alimentaria
DPFADRDSYKHSPPYYPAPEIGRVLTDRRWRTAMNAAQSLVANMTLYEKVNITSGTGWEMGPCVGNTGTVERLGIKSICLQDGPLGIRFADKITAFPAGITIASTFSRKLVQERGAAMGRENRRKGVDITLSPVVGPLGRHANGGRIWEGFSADPYLAGKLAAQAVEGIQSQNVMAVVKHMVGNEQEHFRQLGEWQGFGFKDLKQPLSSNIDDRTMNEAYLWPFADAVRANVGSVMCSYQQINGSQGCQNSALLNGKLKEEFGFQGFVMSDWLAQRSGVASVLAGLDMSMPGDGLVWADGIPLMGYELTKMVLNGTVEQSRVDDMVTRILTPLLYLSITPGNPNFSSWTNETTGLQFPGADRGPNITVNQHVEVRDSYTTKAALDGATAALVLLKNVQSALPLKNVTKLNIFGVGSTTGPLGAVCGENMQCSDGALILGWGSGSVYPSDYQSPYEAIKERAEQDKIQVGGTTESWGNLTRVEQLAAESDASIVFVLSDSGESTGIVDGNIGDRNNLTLWHNGDEVVRTVASQSANTIVVVTTVGPVNLEKWINHPNVTAVLLTGPAGDFGGKAAASVLFGDVAPSGKLPFTIARNDTDYVPLTTKVPSDGLPQDFFTEGTLLDYKRFDALKKTPRFEFGYGLSYSNITLGNLQAKFAFNNIPEFLPTPFPPLDPHKPANVFPTELNGSVFPSDIDPIPKYVYPYLNSTSELNSNISRYPYPEGFSVVQSNGTNINGGAVGGNPALWLTALYVAHSISNHGPYDTGVVTQLYIAFPQDNEGLQTAPRQLRGFERSELPVGEQRGVQYDIQWRDLAVWDVSIQSWRVQRGDYEIFVGHSSRDLVLATKFTLN

SEQ ID NO: 16 - DNA, 37 bp, Artificial Sequence
  Primer
cgggatcccgcgatgatcttctctctgcaactactac

SEQ ID NO: 17 - DNA, 33 bp, Artificial Sequence
  Primer
cgcctaggctacaaagtgaaagtctcacatagc

SEQ ID NO: 18 - DNA, 30 bp, Artificial Sequence
  Primer
cccaagcttgggtttggagggggtgaaaaa

SEQ ID NO: 19 - DNA, 38 bp, Artificial Sequence
  Primer
cccaagcttgggctaaagacctaaccaattcttagtct

SEQ ID NO: 20 - DNA, 31 bp, Artificial Sequence
  Primer
cgggatcccgcgatgattgcaaaaatacccc

SEQ ID NO: 21 - DNA, 30 bp, Artificial Sequence
  Primer
cgcctaggctactggagagtaaaggactcg

SEQ ID NO: 22 - DNA, 31 bp, Artificial Sequence
  Primer
cgggatcccgcgatgctcgcattcgtcctac

SEQ ID NO: 23 - DNA, 32 bp, Artificial Sequence
  Primer
cgggatcccgctacttgagagtgaagctggtg

SEQ ID NO: 24 - DNA, 33 bp, Artificial Sequence
  Primer
cgggatcccgcgatggctccacccccgcctcct

SEQ ID NO: 25 - DNA, 33 bp, Artificial Sequence
  Primer
cgcctaggttaagcaatcgtgatgcgaccaagg

SEQ ID NO: 26 - DNA, 32 bp, Artificial Sequence
  Primer
cgcctaggcgcgatggaggaattatcggaggc

SEQ ID NO: 27 - DNA, 29 bp, Artificial Sequence
  Primer
cgcctaggctaccggctgaacttctcttc

SEQ ID NO: 28 - DNA, 69 bp, Artificial Sequence
  Primer
cgcctaggttaatgatggtgatgatggtggctgccgcgcggcaccagcctaggcaaagtgaaagtctca

SEQ ID NO: 29 - DNA, 76 bp, Artificial Sequence
  Primer
cccaagcttgggttaatgatggtgatgatggtggctgccgcgcggcaccagcctaggaagacctaaccaattctta

SEQ ID NO: 30 - DNA, 68 bp, Artificial Sequence
  Primer
cgcctaggttaatgatggtgatgatggtggctgccgcgcggcaccagcctaggctggagagtaaagga

SEQ ID NO: 31 - DNA, 70 bp, Artificial Sequence
  Primer
cgggatcccgttaatgatggtgatgatggtggctgccgcgcggcaccagcctaggcttgagagtgaagct

SEQ ID NO: 32 - DNA, 67 bp, Artificial Sequence
  Primer
cgcctaggttaatgatggtgatgatggtggctgccgcgcggcaccagcctaggagcaatcgtgatgc

SEQ ID NO: 33 - DNA, 68 bp, Artificial Sequence
  Primer
cgcctaggttaatgatggtgatgatggtggctgccgcgcggcaccagcctaggctgaacttctcttcc

SEQ ID NO: 34 - protein, 16 aa, Yarrowia lipolytica
MIFSLQLLLTTALAAS

SEQ ID NO: 35 - protein, 16 aa, Yarrowia galli
MIFSLQLLLTTVPVLA

SEQ ID NO: 36 - protein, 17 aa, Yarrowia lipolytica
MLAFVLLLTMLLAAALA

SEQ ID NO: 37 - protein, 20 aa, Yarrowia galli
MLAFVLLLTTLQALLTAVLA

SEQ ID NO: 38 - protein, 17 aa, Yarrowia yakushimensis
MLAFVLLLTSLLTAVLA

SEQ ID NO: 39 - protein, 16 aa, Yarrowia alimentaria
MFSLLLLLTLLSTALA

(19) United States
(12) Patent Application Publication
(10) Pub. No.: US 2018/0044653 A1
(43) Pub. Date: Feb. 15, 2018

(54) Mutant Yeast Strain Capable of Degrading Cellobiose

(71) Applicants: Institut National de la Recherche Agronomique, Paris (FR); Institut National des Sciences Appliquees de Toulouse, Toulouse (FR); Centre National de la Recherche Scientifique, Paris (FR)

(72) Inventors: Alain Marty, Toulouse (FR); Zhongpeng Guo, Toulouse (FR); Sophie Duquesne, Toulouse (FR); Sophie Bozonnet, Toulouse (FR); Jean-Marc Nicaud, Trappes (FR); Michael O'Donohue, Toulouse (FR)

(21) Appl. No.: 15/558,221

(22) PCT Filed: Apr. 22, 2016
(86) PCT No.: PCT/EP2016/059079

§ 371 Date: Sep. 14, 2017

(30) Foreign Application Priority Data
Apr. 23, 2015 (EP) 15164928.2

(51) Int. Cl. C12N 9/42; C12P 7/64; C12P 19/02; C12N 15/62; C12N 15/81

(52) U.S. Cl. CPC C12N 9/2445; C12N 15/625; C12N 15/815; C12P 19/02; C12P 7/6463; C12P 7/649; C12Y 302/01021

(57) ABSTRACT

The invention relates to a method for obtaining a mutant oleaginous yeast strain capable of growing on cellobiose as carbon source, comprising overexpressing in said strain two β-glucosidase enzymes further comprising a N-terminal signal peptide. The invention also relates to a mutant yeast strain obtained by said method.

The present invention relates to mutant yeast strains, such as Yarrowia lipolytica strains, capable of growing on cellobiose as carbon source and means for obtaining such mutant strains.

It is widely recognized that lignocellulosic biomass (or LC biomass) will form an important part of the future bio-economy. However, the use of this renewable resource as feedstock for industrial activities poses a major challenge, because its deconstruction to sugars and lignin is complex, requiring a series of unit operations. These include costly pretreatment and enzyme hydrolysis steps, the latter requiring the action of several types of enzymes (Pedersen and Meyer, 2010; Wilson, 2009). Indeed, the hydrolysis of cellulose alone requires the synergistic action of endoglucanases (EC 3.2.1.4), cellobiohydrolases (EC 3.2.1.91) and β-glucosidases (EC 3.2.1.21) (Tornme and Warren, 1995). Endoglucanases are active on the internal bonds in cellulose and release free reducing and non-reducing extremities, which are used by cellobiohydrolases as starting points for exo-processive hydrolysis that yields cellodextrins as products. Finally, β-glucosidases convert cellodextrins into glucose (Sun and Cheng, 2002).

One strategy to reduce investment and operational costs in LC biomass processing is to internalize enzyme production and combine enzyme hydrolysis with fermentation. This is known as consolidated bioprocessing (or CBP) and can be achieved using a microorganism that possesses the dual ability to produce biomass-hydrolyzing enzymes and ferment sugars to products of commercial interest, thus allowing a one-pot type bioconversion process in which process integration is maximized (Lynd et al., 2005). While CBP is considered to be an ultimate aim for biorefining, the ways to achieve this goal are not simple. By way of example, efforts at engineering Yarrowia lipolytica able to degrading xylose—which is not reported as being naturally consumed by Y. lipolytica—by overexpressing endogenous putative genes involved in xylose degrading pathway, were not successful (see International Application WO 2013/192520). Although the number of naturally-occurring, biomass-degrading microorganisms is no doubt large, those that possess the ability to hydrolyze LC biomass and ferment free sugars into desired products, such as ethanol, butanol, hydrogen, fatty acid ethyl esters (FAEE) or isopropanol, at industrially-compatible rates and titers, are probably very rare and so far undiscovered (La Grange, 2010). Additionally, many of the best known biomass-degrading microorganisms display low β-glucosidase (cellobiase) activity, meaning that the hydrolysis of cellobiose constitutes a rate-limiting step during the enzymatic processing of cellulose (Duff, 1985; Holtzapple et al., 1990; Stockton et al., 1991). Therefore, engineering cellobiose-degrading ability into microorganisms is a vital step towards the development of cellulolytie biocatalysts suitable for CBP. In this respect, examples of recent work performed on Saccharomyces cerevisiae, the current workhorse of biotechnological processes, are noteworthy (Lee et al., 2013; Lian et al., 2014; Nan et al., 2014). In these studies, even though the engineered S. cerevisiae strains exhibited poor cellulose-degrading ability, the fact that they both produce significant cellobiase activity means that their incorporation into a simultaneous saccharification and fermentation (SSF) process is likely to reduce the loading of external cellulases and thus overall process cost (Lee et al., 2013).

Although ethanol is the target molecule in many biorefinery concepts, Fatty Acid Esters (FAEs) such as those used in biodiesel, are also attractive targets. This is because FAEs display high energy density and are well-tolerated by production strains (Zhang et al., 2012). Currently, FAEs arc mainly produced by transesterification of plant oils using an alcohol (methanol or ethanol) and base, acid or enzyme catalysts (Demirba, 2003). However, the high cost of this process and various issues surrounding the production of plant oils for non-food purposes makes the search for alternative routes both attractive and strategically pertinent. In this respect, microbial production of biofuels (so-called microdiesel and microkerosene) represents a sustainable and quite economical way to produce FAEs. For this purpose, both Esherichia coli and S. cerevisiae have been engineered to produce structurally-tailored fatty esters (Steen et al., 2010; Shi et al., 2012; Runguphan and Keasling, 2014). However, neither of these microorganisms is naturally able to accumulate high amounts of lipids, nor are they able to degrade cellulose. Moreover, in these microorganisms the biosynthesis of fatty acid (FA) is highly regulated (Nielsen, 2009), thus limiting the possibility to improve lipid production (Runguphan and Keasling. 2014; Shi et al., 2012; Valle-Rodriguez et al., 2014).

So-called oleaginous microorganisms, which naturally accumulate lipids to more than 20% of their dry cell weight (DCW) (Ratledge, 2005; Thevenieau and Nicaud, 2013), have already been exploited for the production of commercially-useful lipids, such as substitutes for cocoa butter and polyunsaturated fatty acids (Papanikolaou and Aggelis, 2010). Therefore, it is unsurprising that microbial lipid or single cell oil (SCO) is also being considered for biodiesel production, especially because this route implies shorter production times, reduced labor costs and simpler scale-up (Easterling et al., 2009). Prominent among the oleaginous microorganisms, Yarrowia lipolytica has been extensively studied and is known to accumulate lipids up to 50% of its dry weight depending on culture conditions (Blazeck et al., 2014; Ratledge, 2005; Thevenieau and Nicaud, 2013). Advantageously, since Y. lipolytica is already widely used in the detergent, food, pharmaceutical, and environmental industries, it has been classified by the FDA (Food and Drug Administration) as “Generally Recognized as Safe” (GRAS) for numerous processes (Groenewald et al., 2014). Nevertheless, despite these advantages, Y. lipolytica displays limited ability for sugar use and is unable to use cellulose or cellobiose as carbon source (Michely et al., 2013), while its genome comprises 6 predicted β-glucosidase genes (BGLs) (Wei et al., 2014). However, in the absence of biochemical data it is impossible to assert that these 6 predicted β-glucosidase genes actually encode β-glucosidases, since family GH3 contains glycoside hydrolases that display other specificities and also because Y. lipolytica does not grow on cellobiose and has not been found to express a detectable level of β-glucosidase activity.

In a recent paper, the use of cellobiose by Y. lipolytica was tackled for the first time, thus opening the way towards the development of an efficient yeast-based CBP microorganism capable of consuming cellulose-derived glucose and converting it into lipids and derivatives thereof (Lane et al., 2014).

The inventors have shared this aim (i.e., providing a cellobiose-degrading mutant Y. lipolytica), but have employed a different strategy that relies upon the activation of endogenous β-glucosidase activity. The inventors have identified two genes, BGL1 (YALIF16027g) and BGL2 (YALI0B14289g), encoding active β-glucosidases in Y. lipolytica, referred to as SEQ ID NO: 4 (YALI_BGL1) and SEQ ID NO: 6 (YALI _BGL2) respectively. The two active β-glucosidases, one of which was mainly cell-associated while the other was present in the extracellular medium, were purified and characterized. The specific growth rate of mutant Y. lipolytica co-expressing BGL1 (wherein the coding sequence is referred to as SEQ ID NO: 3) and BGL2 (wherein the coding sequence is referred to as SEQ ID NO: 5) on cellobiose was 0.16 h⁻¹, similar to that of the control grown on glucose in defined media. Significantly, Y. lipolytica Δpox co-expressing both BGLs grew better than the strains expressing single BGLs in simultaneous saccharification and fermentation on cellulose.

In addition, the comparison of the specific activities of YALI_BGL1 (also referred to as Bgl1) and YALI_BGL2 (also referred to as Bgl2) on cellobiose (108 units/mg and 25 units/mg protein respectively) with that of the commercially available β-glucosidase from Aspergillus niger (5.2 units/mg protein), the enzyme that is generally used to complement the cellulolytic cocktail of T. reesei (Yan and Lin, 1997), is rather flattering for the former. Moreover, the K_{M} values describing the cellobiolytic reactions catalyzed by Bgl1 and Bgl2 are approximately 10 and 4-fold lower than those of the β-glucosidases from S. fibuligera (2.8 mM Bgl1) and A. niger (2.7 mM) (Yan and Lin, 1997), meaning that the minimum concentration of cellobiose required for effective catalysis to occur is much lower. Likewise, comparing the apparent performance constants, k_{cat}/K_{M}, of Y. lipolytica Bgls with those of other reported β-glucosidases (Belancic et al., 2003; Daroit et al., 2008; Galas and Romanowska, 1996; Gonzalez-Pombo et al., 2008; Leclerc et al., 1987; Machida et al., 1988; Yan and Lin, 1997) suggests that the enzymes described in this study hydrolyze cellodextrins more efficiently.

The bi-functional Y. lipolytica (expressing BGL1 and BGL2) is of an interest for biotechnological processes related to lipid production from lignocellulosic biomass. In addition, overexpression of BGL1 and BGL2 in oleaginous yeast strains other than Y. lipolytica is also of an interest for biotechnological processes.

Accordingly, the present invention provides a method for obtaining an oleaginous yeast strain capable of growing on cellobiose as carbon source, wherein said method comprises overexpressing in said strain a β-glucosidase (EC 3.2.1.21) having at least 80% identity, or by order of increasing preference at least 83%, 85%, 90%, 92%, 95%, 96%, 97%, 98% or 99% identity, with the polypeptide of sequence SEQ ID NO: 1 (mature YALI_BGL1) further comprising a N-terminal signal peptide and a β-glucosidase (EC 3.2.1.21) having at least 80% identity, or by order of increasing preference at least 82%, 85%, 90%, 92%, 95%, 96%©, 97%, 98% or 99% identity, with the polypeptide of sequence SEQ ID NO: 2 (mature YALI_BGL2) further comprising a N-terminal signal peptide.

The term overexpressing a β-glucosidase in a yeast strain, herein refers to artificially increasing the quantity of said β-glucosidase produced in a yeast strain compared to a reference (control) yeast strain (wherein said β-glucosidase is not overexpressed). This term also encompasses expression of a β-glucosidase in a yeast strain which does not naturally contain a gene encoding said β-glucosidase.

An advantageous method for overexpressing both β-glucosidases comprises introducing into the genome of said yeast strain DNA constructs comprising a nucleotide sequence encoding said β-glucosidases, placed under the control of a promoter.

Nucleotide sequences encoding YALI_BGL1 and YALI_BGL2 are provided in SEQ ID NO: 3 and SEQ ID NO: 5 respectively.

Unless otherwise specified, the percent of identity between two sequences which are mentioned herein is calculated from an alignment of the two sequences over their whole length, not comprising the signal sequence. One can use the BLAST program (Tatusova and Madden T L, 1999) with the default parameters (open gap penalty=2; extension gap penalty=5; matrix=BLOSUM 62).

The signal peptide (or signal sequence) drives secretion of the β-glucosidase into the extracellular space (e.g., periplasm, external medium) of the yeast. After secretion, the signal peptide is usually cleaved (removed) by a peptidase leading to a mature β-glucosidase. The signal peptide can further allow glycosylation in the case of β-glucosidase bearing potential sites of glycosylation.

Signal peptides are well known in the art (see for review von Heijne, 1985). In particular, signal peptides able to drive secretion of a protein into the extracellular space in yeast and methods to perform such a secretion are well known in the art (see Sreekrishna et al., 1997; Hashimoto of al., 1998; Koganesawa et al.; 2001; Gasmi et al., 2011; Madzak and Beckerich, 2013). Methods for identifying a signal peptide (signal sequence) are also well known in the art. One can use the programs Signal-BLAST (Franck and Sippl, 2008) and/or SignalP 4.1 Server (Petersen et al., 2011).

The signal peptide can be from a protein from any organism, such as mammal, bacteria, yeast, preferably from a protein form a yeast, more preferably from Yarrowia.

The signal peptide of the β-glucosidase having at least 80% identity with the polypeptide of sequence SEQ ID NO: 1 and the signal peptide of the β-glucosidase having at least 80% identity with the polypeptide of sequence SEQ ID NO: 2 can be identical or different, in terms of space of secretion (periplasm or external medium) and/or in terms of amino acid sequence. By way of example, the signal peptide of the β-glucosidase having at least 80% identity with the polypeptide of sequence SEQ ID NO: 1 drives secretion of said β-glucosidase into the periplasm space or the external medium and the signal peptide of the β-glucosidase having at least 80% identity with the polypeptide of sequence SEQ ID NO: 2 drives secretion of said β-glucosidase into the periplasm space or the external medium.

Advantageously, the signal peptide of the β-glucosidase having at least 80% identity with the polypeptide of sequence SEQ ID NO: 1 drives secretion of said β-glucosidase into the periplasm space. It can be chosen from the signal peptides SEQ ID NO: 34 or SEQ ID NO: 35.

Advantageously, the signal peptide of the β-glucosidase having at least 80% identity with the polypeptide of sequence SEQ ID NO: 2 drives secretion of said β-glucosidase into the external medium. It can be chosen from the signal peptides SEQ ID NO: 36, SEQ ID NO: 37, SEQ ID NO: 38 or SEQ ID NO: 39.

The signal, peptide can be the naturally occurring signal sequence of the β-glucosidase to overexpress or a modified signal sequence, such as the Yarrowia alkaline extracellular protease (Aep; Fabre et al., 1991) or the extracellular lipase (Lip2p) signal sequences (Pignède et al., 2000; Nicaud et al., 2002).

Methods for determining the presence of a signal sequence (signal peptide) in a protein are well known in the art. One can use the programs Signal-BLAST (Franck and Sippl, 2008) and/or SignalP 4.1 Server (Petersen et al., 2011).

Methods for determining whether an enzyme has a β-glucosidase activity (EC 3.2.1.21) are known in the art. By way of example, β-glucosidase activity can be measured by quantifying the release of pNP (p-nitrophenol) from pNPGlc as described in Guo et 2011.

The β-glucosidase having at least 80% identity with the polypeptide of sequence SEQ ID NO: 1 (mature YALI_BGL1) can be a heterologous or endogenous β-glucosidase of the oleaginous yeast strain.

Advantageously, the β-glucosidase having at least 80% identity with the polypeptide of sequence SEQ ID NO: 1 (mature YALI_BGL1) is from a yeast strain, preferably an oleaginous yeast strain, such as Candida, Cryptoccocus, Lipomyces, Rhodosporidium (e.g., Rhodosporidium toruloides), Rhodotorula (e,g., Rhodotorula glutinis), Trichosporon or Yarrowia, more preferably a Yarrowia strain. According to this embodiment, the Yarrowia strain is preferably selected from Y. lipolytica and Y. galli, more preferably a Y. lipolytica strain.

In a preferred embodiment, the β-glucosidase having at least 80% identity with the polypeptide of sequence SEQ ID NO: 1 (mature YALI_BGL1) is selected from the group consisting of the β-glucosidase of SEQ ID NO: 1 (mature YALI_BGL1), and SEQ ID NO: 8 (YAGA_BGL1 without its naturally occurring N-terminal signal sequence), preferably SEQ ID NO: 1 (mature YALI_BGL1).

In another preferred embodiment, the β-glucosidase having at least 80% identity with the polypeptide of sequence SEQ ID NO: 1 (mature YALI_BGL1) further comprising a N-terminal signal peptide is selected from the group consisting of SEQ ID NO: 4 (YALI_BGL1; BGL1 from Yarrowia lipolytica CLIB122, comprising its naturally occurring N-terminal signal sequence) and SEQ ID NO: 7 (YAGA_BGL1; BGL1 from Yarrowia galli CBS 9722, comprising its, naturally occurring N-terminal signal sequence), preferably SEQ ID NO: 4.

The β-glucosidases included in sequences SEQ ID NO: 4 (YALI_BGL1) and SEQ ID NO: 7 (YAGA_BGL1) have respectively 100% and 84.08% identity with the polypeptide of sequence SEQ ID NO: 1 (mature YALI_BGL1).

The β-glucosidase having at least 80% identity with the polypeptide of sequence SEQ ID NO: 2 (mature YALI_BGL2) can be a heterologous or endogenous β-glucosidase of the oleaginous yeast strain.

Advantageously, the β-glucosidase having at least 80% identity with the polypeptide of sequence SEQ ID NO: 2 (mature YALI_BGL2) is from a yeast strain, preferably an oleaginous yeast strain, such as Candida, Cryptoccocus, Lipomyces, Rhodosporidium (e.g., Rhodosporidium toruloides), Rhodotorula (e.g., Rhodotorula Trichosporon or Yarrowia, more preferably a Yarrowia strain. According to this embodiment, the Yarrowia strain is preferably selected from a Y. lipolytica, Y. galli, Y. yaktishirnensis or Yarrowia alimentaria strain, more preferably a Y. lipolytica strain.

In another preferred embodiment, the β-glucosidase having at least 80% identity with the polypeptide of sequence SEQ ID NO: 2 (mature YALI_BGL2) has the amino acid sequence SEQ ID NO: 9. This sequence SEQ ID NO: 9 corresponds to the consensus amino acid sequence obtained from mature YALI_BGL2, YAGA_BGL2, YAYA_BGL2 and YAAL_BGL2 (as described above).

In another preferred embodiment, the β-glucosidase having at least 80% identity with the polypeptide of sequence SEQ ID NO: 2 (mature YALI_BGL2) is selected from the group consisting of the β-glucosidase of SEQ ID NO: 2 (mature YALI_BGL2), SEQ ID NO: 11 (YAGA_BGL2 without its naturally occurring N-terminal signal sequence), SEQ ID NO: 13 (YAYA_BGL2 without its naturally occurring N-terminal signal sequence) and SEQ ID NO: 15 (YAAL_BGL2 without its naturally occurring N-terminal signal sequence), preferably SEQ ID NO: 2 (mature YALI_BGL2).

In another preferred embodiment, the β-glucosidase having at least 80% identity with the polypeptide of sequence SEQ ID NO: 2 (mature YALI_BGL2) further comprising a N-terminal signal peptide is selected from the group consisting of SEQ ID NO: 6 (YALI_BGL2; BGL2 from Yarrowia lipolytica CLIB122, comprising its naturally occurring N-terminal signal sequence), SEQ ID NO: 10 (YAGA_BOL2; BGL2 from Yarrowia galli CBS 9722, comprising its naturally occurring N-terminal signal sequence), SEQ ID NO: 12 (YAYA_BGL2; BGL2 from Yarrowia yakushimensis CBS 10253, comprising its naturally occurring N-terminal signal sequence) and SEQ ID NO: 14 (YAAL_BGL2; BGL2 from Yarrowia alimentaria CBS 10151, comprising its naturally occurring N-terminal signal sequence), preferably SEQ ID NO: 6.

The β-glucosidases included in sequences SEQ ID NO: 6 (YALI_BGL2), SEQ ID NO: 10 (YAGA_BGL2), SEQ ID NO: 12 (YAYA_BGL2) and SEQ ID NO: 14 (YAAL_BGL2) have respectively 100%, 93.55%, 85.21% and 82.14% identity with the polypeptide of sequence SEQ ID NO: 2 (mature YALI_BGL2).

In another preferred embodiment, both β-glucosidases (the β-glucosidase having at least 80% identity with the polypeptide of sequence SEQ ID NO: 1 and the β-glucosidase having at least 80% identity with the polypeptide of sequence SEQ ID NO: 2) are from a yeast strain, preferably an oleaginous yeast strain, such as Candida, Cryptoccocus, Lipomyces, Rhodosporidium (e.g. Rhodosporidium toruloides), Rhodotorula (e.g., Rhodotorula glutinis), Trichosporon or Yarrowia, more preferably a Yarrowia strain. According to this embodiment, the Yarrowia strain is preferably selected from a Y. lipolytica or Y. galli strain, and more preferably a Y. lipolytica strain.

Oleaginous yeast strains, which naturally accumulate lipids to more than 20% of their dry cell weight, are well known in the art (Ratledge, 1994 and 2005). They include the genus Candida, Cryptoccocus, Lipomyces, Rhodosporidium (e.g., Rhodosporidium toruloides), Rhodotorula (e.g., Rhodotorula glutinis), Trichosporon and Yarrowia.

In a preferred embodiment, the oleaginous yeast strain is a Yarrowia strain, preferably a Yarrowia lipolytica strain.

Advantageously, said yeast strain is auxotrophic for leucine (Leu-) and optionally for the decarboxylase orotidine-5′-phosphate (Ura-).

Said yeast can also be a mutant yeast strain wherein the expression or activity of the endogenous isoforms of acyl-coenzymeA oxidases (AOX, EC 6.2.1.3) involved, at least partially, in the β-oxidation of fatty acids, is inhibited. In yeasts, 6 genes (POX1, POX2, POX3, POX4, POX5 and POX6) encode these isoforms. Said inhibition of the expression or activity can be total or partial. Total or partial inhibition of the expression or activity of these enzymes leads to accumulation by yeast of dodecanedioic acid without use of accumulated fat. More particularly, the coding sequence of the genes POX1-6 and the peptide sequence of AOX1-6 from Y. lipolytica CLIB122 are available in either at Genolevures database (http://genolevures.org/) or at GRYC database (http://gryc.inra.fr/) or in GenBank database under the following accession numbers or names: POX1/AOX1=YALI0E32835g/YALI0E32835p, POX2/AOX2=YALI0F10857g/YALI0F10857p; POX3/AOX3=YALI0D24750g/YALI0D24750p; POX4/AOX4=YALI0E27654g/YALI0E27654p; POX5/AOX5=YALI0C23859g/YALI0C23859p; POX6/AOX6=YALI0E06567g/YALI0E06567p. The peptide sequences of the acyl-CoA oxidases of Y. lipolytica have 45% identity or 50% similarity with those from other yeasts. The degree of identity between the acyl-CoA oxidases varies from 55% to 70% (or from 65 to 76% similarity) (see International Application WO 2006/064131). A method of inhibiting the expression of the 6 endogenous AOX in a Y. lipolytica strain is described in Beopoulos et al., 2008 and International Applications WO 2006/064131, WO 2010/004141 and WO 2012/001144.

The yeast strain can further comprise other mutations such as those described in International Applications WO 2006/064131, WO 2010/004141, WO 2012/001144, WO 2014/178014 and WO 2014/136028 which are useful for obtaining a fatty acids producing yeast strain.

In particular, the yeast strain can be genetically modified to improve lipid accumulation. Said yeast strain having improved properties for lipid accumulation can be a mutant yeast strain, preferably a Y. lipolytica mutant strain, wherein at least one protein, preferably at least one endogenous protein, selected from the group consisting of an acyl-CoA:diacylglycerol acyltransferase 2 (encoded by DGA1), an acyl-CoA:diacylglycerol acyltransferase 1 (encoded by DGA2), a glycerol-3-phosphate dehydrogenase NAD+ (encoded by GPD1), an acetyl-CoA carboxylase (encoded by ACCT) and a hexokinase (encoded by HXK1) is overexpressed, and/or the expression or activity of at least one endogenous protein selected from the group consisting of the glycerol 3-phosphate dehydrogenase (encoded by GUT2), the triglyceride lipase (encoded by TGL4) and the peroxin 10 (encoded by PEX10) is inhibited.

Advantageously, the 5 proteins, acyl-CoA:diacylglycerol acyltransferase 2, acyl-CoA:diacylglycerol acyltransferase 1, glycerol-3-phosphate dehydrogenase NAD+, acetyl-CoA carboxylase and hexokinase, are overexpressed, and the expression or activity of the 3 endogenous proteins, glycerol 3-phosphate dehydrogenase, triglyceride lipase and peroxyne, are inhibited in said mutant yeast strain.

In another advantageous embodiment, said yeast strain—preferably a Yarrowia strain, more preferably a Y. lipolytica strain—having improved properties for lipid accumulation is a mutant yeast strain in which the expression or activity of the endogenous isoforms of acyl-coenzymeA oxidases (AOX, EC 6.2.1.3) involved, at least partially, in the β-oxidation of fatty acids (e.g., POX1 to POX6 in Y. lipolytica) and the triglyceride lipase (encoded by TGL4) are inhibited, and an acyl-CoA:diacylglycerol acyltransferase (encoded by DGA2) and a glycerol-3-phosphate dehydrogenase (encoded by GPD1)—preferably the endogenous DGA2 and GPD1—are overexpressed.

A method of overexpressing the endogenous genes DGA1, DGA2, GPD1 and ACC1 and inhibiting the expression or activity of the endogenous genes GUT2, TGL4 and PEX10 in a Y. lipolytica strain is described in International Application WO 2014/136028.

A method of overexpressing the endogenous genes DGA2, GPD1 and HXK1, and inhibiting the expression or activity of the endogenous gene TGL4 in a Y. lipolytica strain is described in Lazar et al., 2014.

Overexpression of a β-glucosidase (endogenous, ortholog, heterologous) in a yeast strain according to the present invention can be obtained in various ways by methods known per se.

Overexpression of a β-glucosidase as defined in the present invention may be performed by placing one or more (preferably two or three) copies of the open reading frame (ORF) of the sequence encoding said β-glucosidase under the control of appropriate regulatory sequences. Said regulatory sequences include promoter sequences, located upstream (at 5′ position) of the ORF of the sequence encoding said β-glucosidase, and terminator sequences, located downstream (at 3′ position) of the ORF of the sequence encoding said β-glucosidase.

Promoter sequences that can be used in yeast are well known to those skilled in the art and may correspond in particular to inducible or constitutive promoters. Examples of promoters which can be used according to the present invention, include the promoter of a Y. lipolytica gene which is strongly repressed by glucose and is inducible by the fatty acids or triglycerides such as the promoter of the POX2 gene encoding the acyl-CoA oxidase 2 (AOX2) of Y. lipolytica and the promoter of the LIP2 gene described in International Application WO 01/83773. One can also use the promoter of the FBA1 gene encoding the fructose-bisphosphate aldolase (see Application US 2005/0130280), the promoter of the GPM gene encoding the phosphoglycerate mutase (see International Application WO 2006/0019297), the promoter of the YAT1 gene encoding the transporter ammonium (see Application US 2006/0094102), the promoter of the GPAT gene encoding the O-acyltransferase glycerol-3-phosphate (see Application US 2006/0057690), the promoter of the TEF gene (Muller et al., 1998; Application US 2001/6265185), the hybrid promoter hp4d (described in International Application WO 96/41889), the hybrid promoter XPR2 described in Mazdak et al. (2000) or the hybrid promoters UAS1-TEF or UAStef-TEF described in Blazeck et al. (2011, 2013, 2014).

Advantageously, the promoter is the promoter of the TEE gene.

Terminator sequences that can be used in yeast arc also well known to those skilled in the art. Example of terminator sequences which can be used according to the present invention include the terminator sequence of the PGK1 gene and the terminator sequence of the LIP2 gene described in International Application WO 01/83773.

The nucleotide sequence of the coding sequences of the heterologous genes can be optimized for expression in yeast by methods well known in the art (see for review Hedfalk, 2012).

Overexpression of an endogenous β-glucosidase can be obtained by replacing the sequences controlling the expression of said endogenous β-glucosidase by regulatory sequences allowing a stronger expression, such as those described above. The skilled person can replace the copy of the gene encoding an endogenous β-glucosidase in the genome, as well as its own regulatory sequences, by genetically transforming the yeast strain with a linear polynucleotide comprising the ORF of the sequence coding for said endogenous β-glucosidase under the control of regulatory sequences such as those described above. Advantageously, said polynucleotide is flanked by sequences which are homologous to sequences located on each side of said chromosomal gene encoding said endogenous β-glucosidase. Selection markers can be inserted between the sequences ensuring recombination to allow, after transformation, to isolate the cells in which integration of the fragment occurred by identifying the corresponding markers. Advantageously also, the promoter and terminator sequences belong to a gene different from the gene encoding the endogenous β-glucosidase to be overexpressed in order to minimize the risk of unwanted recombination into the genome of the yeast strain.

Overexpression of an endogenous β-glucosidase can also be obtained by introducing into the yeast strain of extra copies of the gene encoding said endogenous β-glucosidase under the control of regulatory sequences such as those described above. Said additional copies encoding said endogenous β-glucosidase may be carried by an episomal vector, that is to say capable of replicating in yeast. Preferably, these additional copies are carried by an integrative vector, that is to say, integrating into a given location in the yeast genome (Madzak et al., 2004). In this case, the polynucleotide comprising the gene encoding said endogenous β-glucosidase under the control of regulatory regions is integrated by targeted integration. Said additional copies can also be carried by PCR fragments whose ends are homologous to a given locus of the yeast, allowing integrating said copies into the yeast genome by homologous recombination. Said additional copies can also be carried by auto-cloning vectors or PCR fragments, wherein the ends have a zeta region absent from the genome of the yeast, allowing the integration of said copies into the yeast genome by random insertion as described in Application US 2012/0034652.

Targeted integration of a gene into the genome of a yeast cell is a molecular biology technique well known to those skilled in the art: a DNA fragment is cloned into an integrating vector, introduced into the cell to be transformed, wherein said DNA fragment integrates by homologous recombination in a targeted region of the recipient genome (Orr-Weaver et al., 1981).

Methods for transforming yeast are also well known to those skilled in the art and are described, inter alia, by Ito et al, (1983), Kleve et al., (1983) and Gysler et al., (1990).

Any gene transfer method known in the art can be used to introduce a gene encoding a β-glucosidase. Preferably, one can use the method with lithium acetate and polyethylene glycol described by Gaillardin et al., (1987) and Le Dall et al., (1994).

The present invention also provides means for carrying out said overexpression.

This includes, in particular, recombinant DNA constructs for expressing one or both β-glucosidase(s) as defined above in a yeast cell (e.g., Y. lipolytica strain). These DNA constructs can be obtained and introduced in said yeast strain by the well-known techniques of recombinant DNA and genetic engineering.

Recombinant DNA constructs of the invention include in particular expression cassettes, comprising a polynucleotide encoding one or both β-glucosidase(s) as defined above, under the control of a promoter functional in yeast cell as defined above.

The expression cassettes generally also include a transcriptional terminator, such as those describes above. They may also include other regulatory sequences, such as transcription enhancer sequences.

Recombinant DNA constructs of the invention also include recombinant vectors containing an expression cassette comprising a polynucleotide encoding one or both β-glucosidase(s) as defined above, under transcriptional control of a suitable promoter.

Recombinant vectors of the invention may also include other sequences of interest, such as, for instance, one or more marker genes, which allow for selection of transformed yeast cells.

The invention also comprises host cells containing a recombinant DNA construct of the invention. These host cells can be prokaryotic cells (such as bacteria cells) or eukaryotic cells, preferably yeast cells.

The invention also provides a method for obtaining a mutant oleaginous yeast strain, preferably a mutant Yarrowia strain (e.g., Y. lipolytica strain), capable of growing on cellobiose as carbon source as defined above, comprising transforming an oleaginous yeast cell with a recombinant DNA construct for expressing both β-glucosidases as defined above or with two recombinant DNA constructs for expressing both β-glucosidases respectively as defined above.

The term “two recombinant DNA constructs for expressing both β-glucosidases respectively as defined above” designates “a recombinant DNA construct for expressing a β-glucosidase having at least 80% identity with the polypeptide of sequence SEQ ID NO: 1 as defined above and a recombinant DNA construct for expressing a β-glucosidase having at least 80% identity with the polypeptide of sequence SEQ ID NO: 2 as defined above”.

The invention also comprises an oleaginous yeast strain, preferably a Yarrowia strain (e.g., Y. lipolytica strain), genetically transformed with a recombinant DNA construct for expressing both β-glucosidases as defined above or with two recombinant DNA constructs for expressing both β-glucosidases respectively as defined above, and overexpressing both β-glucosidases as defined above. In said mutant (transgenic) yeast strain one to two recombinant DNA construct(s) of the invention is/are comprised in a transgene stably integrated in the yeast genome, so that it is passed onto successive yeast generations. Thus the mutant (transgenic) yeast strain of the invention includes not only the yeast cell resulting from the initial transgenesis, but also their descendants, as far as they contain one or two recombinant DNA construct(s) of the invention. The overexpression of both β-glucosidases as defined above in said yeast strains provides them an ability to grow on cellobiose as carbon source, when compared with an oleaginous yeast strain devoid of said transgene(s).

The present invention also comprises a mutant oleaginous yeast strain as defined above, preferably a mutant Yarrowia strain (e.g., Y. lipolytica strain), wherein a β-glucosidase having at least 80% identity with the polypeptide of sequence SEQ ID NO: 1 and a β-glucosidase having at least 80% identity with the polypeptide of sequence SEQ ID NO: 2 are overexpressed. This mutant oleaginous yeast strain is obtainable by a method of the invention and contains one or two recombinant expression cassette(s) of the invention.

The present invention further comprises a mutant oleaginous yeast strain as defined above, preferably a mutant Yarrowia strain (e.g., Y. lipolytica strain) comprising, stably integrated in its genome, a recombinant DNA construct for expressing both β-glucosidases as defined above or two recombinant DNA constructs for expressing both β-glucosidases respectively as defined above.

The present invention also provides the use of a mutant oleaginous yeast strain, preferably a mutant Yarrowia strain (e.g., Y. lipolytica strain), as defined above for producing lipids from lignocellulosic biomass, in particular from cellobiose or cellulose.

As used herein, the term producing lipids refers to the accumulation and optionally secretion of lipids.

The present invention also provides a method of producing lipids, comprising a step of growing a mutant oleaginous yeast strain, preferably a mutant Yarrowia strain (e.g., Y. lipolytica strain), of the invention on a lignocellulosic biomass, in particular on cellobiose or cellulose.

Methods for extracting and purifying lipids produced by cultured yeast strains are well known to those skilled in the art (Papanikolaou et al. 2001, 2002 and 2008; André et al., 2009). For example, the total lipids can be extracted according to the method described by Papanikolaou et al., 2001, and fractionated according to the methods described by Guo et al., 2000 and Fakas et al., 2006.

The present invention also provides an isolated β-glucosidase (EC 3.2.1.21) having an amino acid sequence selected from the group consisting of SEQ ID NO: 7, 8 and 10 to 15.

The present invention also provides the use of an isolated β-glucosidase (EC 3.2.1.21) having an amino acid sequence selected from the group consisting of SEQ ID NO: 7, 8 and 10 to 15 for degrading cellobiose.

The present invention will be understood more clearly from the further description which follows, which refers to non-limitative examples illustrating the overexpression of Bgl1 and Bgl2 in Y. lipolytica.

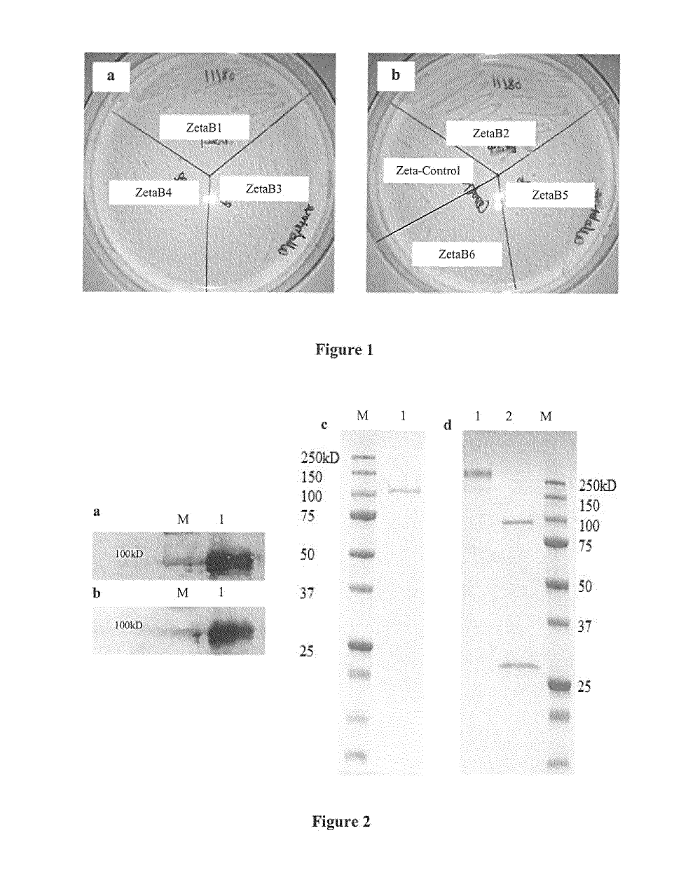

FIG. 1: Screening of Y. lipolytica expressing the 6 putative β-glucosidases on (a) indication plate containing YNBcasa medium supplemented with 1 mM p-nitrophenyl-β-D-glucoside (pNPG), and (b and c) YNBC plate with cellobiose as sole carbon source.

FIG. 2: Western blot detection of the expressed β-glucosidases (a) M, molecular weight standards; lane 1, intracellular Bgl1, and (b) lane 1, extracellular Bgl1, and SDS-PAGE analysis of the purified β-glucosidases from Y. lipolytica JMY1212 transformants (c) lane 1, purified Bgl1-His6, and (d) lane 1, purified Bgl2; lane 2, endo-H treated Bgl2 (The lower band in lane 2 represents the expected size of Endo-H).

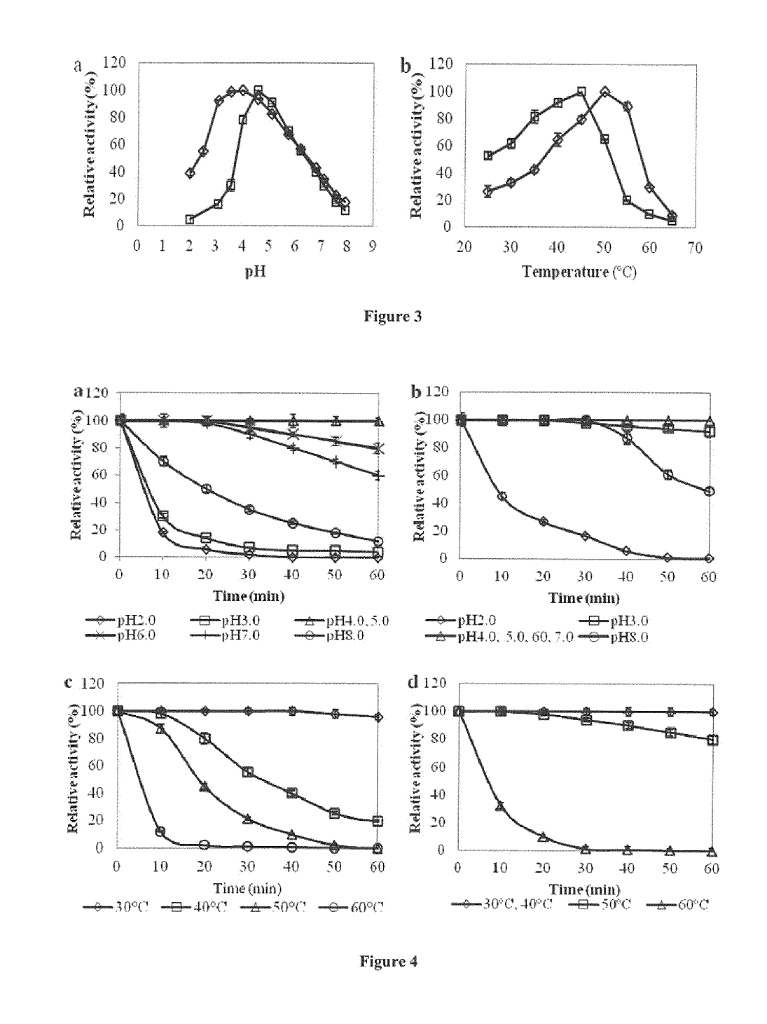

FIG. 3: Optimal pH (a) and temperature (b) of Bgl1 (square) and Bgl2 (diamond) from Y. lipolytica JMY1212. Each data point represents the mean of three independent experiments and the error bar indicates the standard deviation.

FIG. 4: Stability of Bgl1 (a) and Bgl2 (b) from Y. lipolytica JMY1212 at pH from 2.0-8.0 as a function of time at 30° C., and stability of Bgl1 (c) and Bgl2 (d) at temperature from 30° C. to 60° C. as a function of time at pH 5. Each data point represents the mean of three independent experiments and the error bar indicates the standard deviation. Only one curve is giving to represent the stability Bgl2 at pH 4.0, 5.0 and 6.0 (b) and at 30 ° C. and 40° C. (d) as 100% of enzyme activity remained for these conditions.

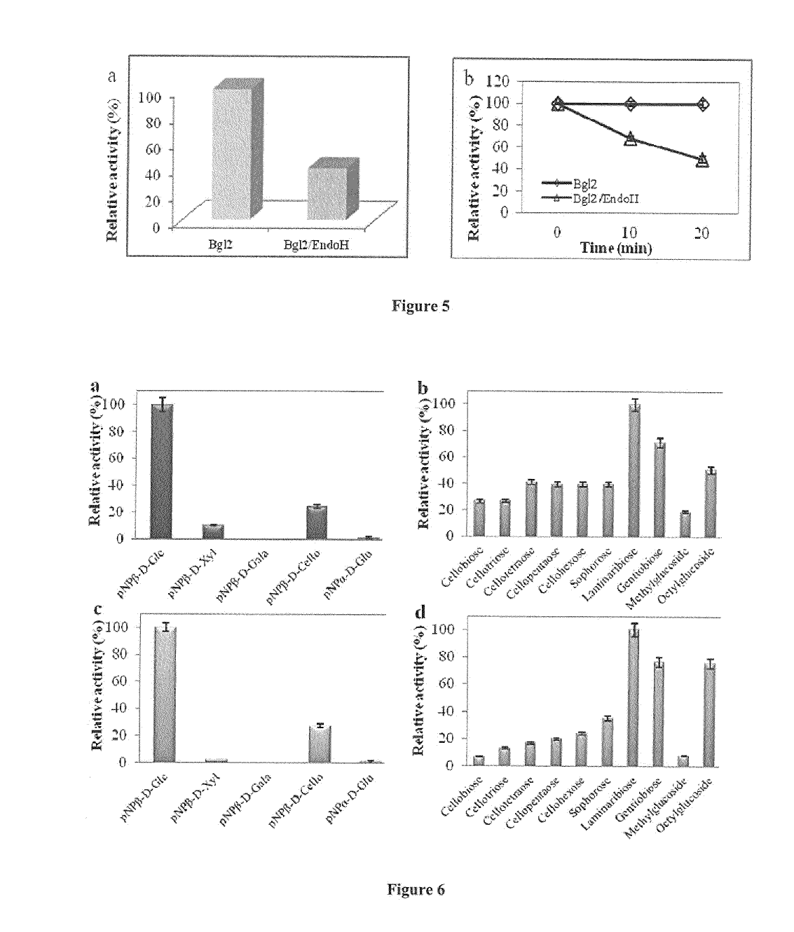

FIG. 5: The hydrolytic activity of Bgl2 on pNPG (a) and the stability of Bgl2 at 40° C. as a function of time at pH5.0 before and after deglycosylation. Each data point represents the mean of three independent experiments and the error bar indicates the standard deviation.

FIG. 6: Comparison of the hydrolytic activity of β-glucosidases from Y. lipolytica JMY1212. Bgl1-His on (a) pNP-derived substrates, and (b) natural glycosyl substrates with different β-configurations; Bgl2 on (c) pNP-derived substrates, and (d) natural glycosyl substrates with different β-configurations.

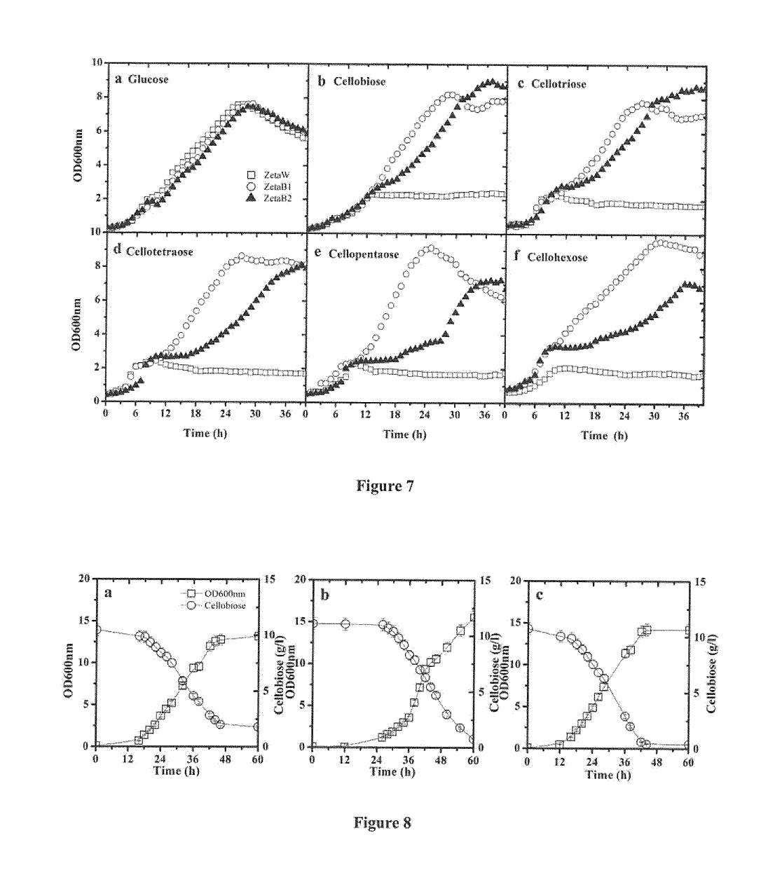

FIG. 7: Comparison of Y. lipolytica ZetaW (control), ZetaB1 (P_{TEF}-BGL1), ZetaB2 (P_{TEF}-BGL2) during aerobic growth on 5 g/L (a) glucose, (b) cellobiose, (c) cellotriose, (d) cellotetraose, (e) cellopentaose and (f) cellohexaose as carbon and energy source. Shown is OD₆₀₀ₙₘ, optical density at 600 nm, versus time. Each data point represents the mean of five independent experiments and the standard deviation is less than 5%.

FIG. 8: Comparison of Y. lipolytica (a) ZetaB1 (P_{TEF}-BGL1), (b) ZetaB2 (P_{TEF}-BGL2) and (c) Zeta-B12 (P_{TEF}-BGL1, P_{TEF}-BGL2) during aerobic growth on 10 g/L cellobiose. Shown are OD₆₀₀ₙₘ, optical density at 600 nm, and cellobiose concentration versus time. Each data point represents the mean of five independent experiments and the error bar indicates the standard deviation.

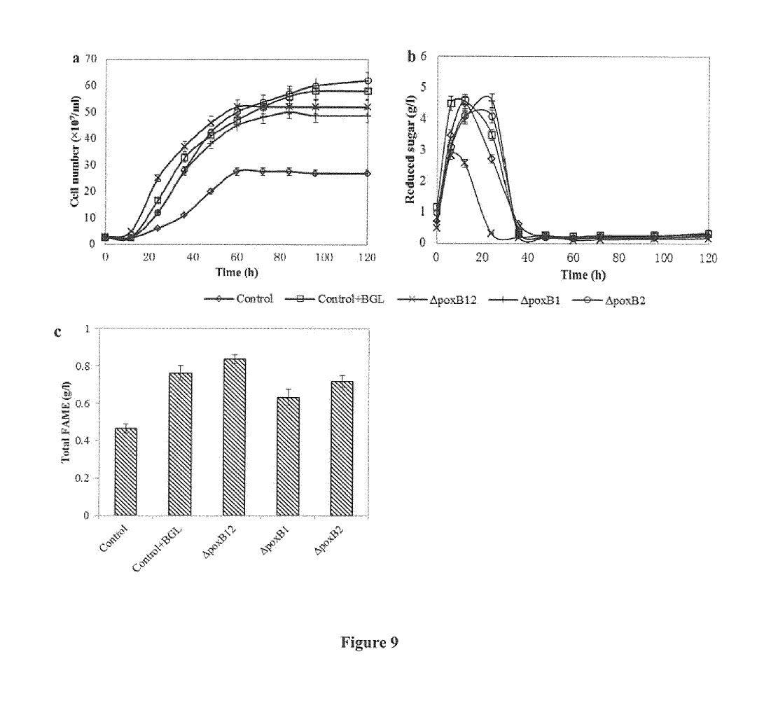

FIG. 9: Growth and lipid production on cellulose medium of Y. lipolytica strains. Growth during SSF on 50 g/L cellulose supplemented with Celluclast 1.5 L. (a) growth expressed as cell number versus time; (b) the concentration of reduced sugar versus time; (c) lipid content at 60 h. Strains are Y. lipolytica ΔpoxB1 (P_{TEF}-BGL1), ΔpoxB2 (P_{TEF}-BGL2) and ΔpoxB12 (P_{TEF}-BGL1, P_{TEF}-BGL2) and ΔpoxW (wild type) under the same condition without (control) or with (control+BGL) extra β-glucosidase (Novozyme 188). Each data point represents the mean of five independent experiments and the error bar indicates the standard deviation.

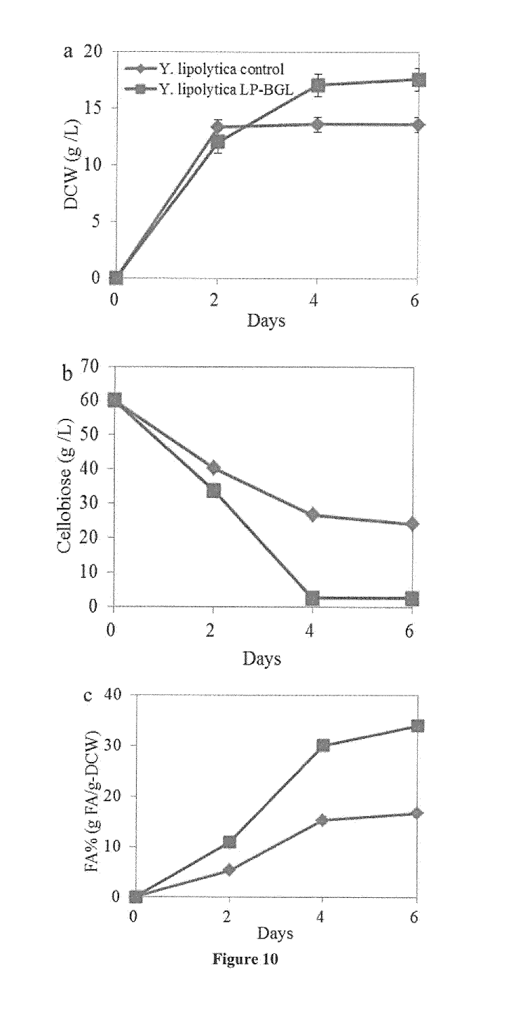

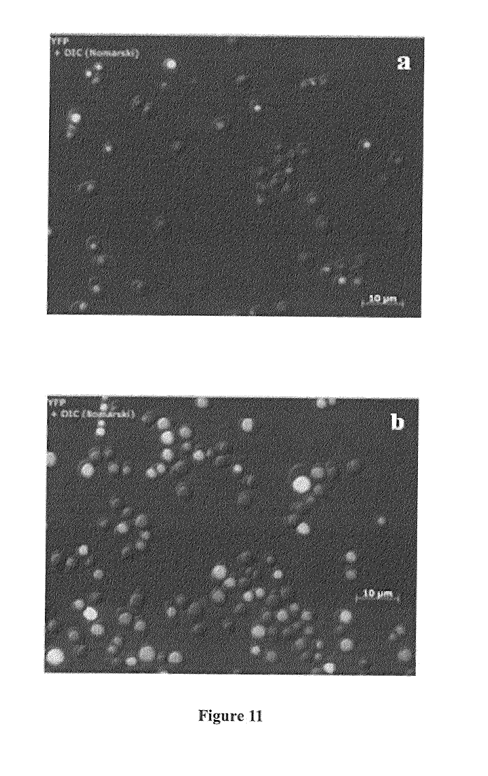

FIG. 10: Growth and lipid production on cellobiose medium of the control Y. lipolytica and Y. lipolytica LP-BGL. (a) Growth expressed as DCW versus time; (b) the concentration of cellobiose versus time; (c) cellular FAs content versus time. Each data point represents the mean of at least three independent experiments and the error bars indicate the standard deviation.

FIG. 11: Visualization of lipid bodies at the end of the lipid production of the control (a) and Y. lipolytica LF-BGL (b). The lipid bodies were stained with Bodipy®.

### EXAMPLE 1

### Cellobiose-Degrading Ability in Yarrowia Lipolytica Stain Using Endogenous Gene Activation

### 1. Materials and Methods

### 1.1 Strains and Media

The genotypes of the microbial strains used in the present study are summarized in Table 1 below.

TABLE 1
Microbial strains used in the present study

| Strains | Relevant genotype | Source of reference |
|---|---|---|
| E. coli DH5 | Φ80dlacZΔm15, recA1, endA1, gyrA96, | Invitrogen |
| | thi-1, hsdR17 (rk⁻, mk⁺), supE44, | |
| | relA1, deoR, Δ(lacZYA-argF) U169 | |
| Y. lipolytica | MATA, ura3-302, leu2-270-LEU2-zeta, | Bordes et al., |
| JMY1212 (Zeta) | xpr2-322 Δlip2, Δlip7, Δlip8 | 2007. |
| Y. lipolytica Δpox | MATA, leu2-270, ura3-302, xpr2-322, pox1-6Δ | Beopoulos et al., |
| JMY1233 | | 2008. |
| ZetaW | MATA, xpr-2-322, Δlip2, Δlip7, Δlip8 | This study |
| ZetaB1 | P_{TEF}-BGL1 | This study |
| ZetaB2 | P_{TEF}-BGL2 | This study |
| ZetaB12 | P_{TEF}-BGL1, P_{TEF}-BGL2 | This study |
| ΔpoxW | MATA, xpr2-322, pox1-6Δ | This study |
| ΔpoxB1 | P_{TEF}-BGL1 | This study |
| ΔpoxB2 | P_{TEF}-BGL2 | This study |
| ΔpoxB12 | P_{TEF}-BGL1, P_{TEF}-BGL2 | This study |

Escherichia coli DH5α was purchased from Invitrogen (Paisley, UK) and used for plasmid construction. The Y. lipolytica strains were routinely cultivated in a medium composed of 1% w/v yeast extract, 1% w/v Bacto peptone, and 1% w/v glucose (YPD), solid media contained 1.5% agar. Transformants were selected on solid YNB medium (0.17% w/v YNB, 1% glucose or cellobiose w/v, 0.5% w/v ammonium chloride, with (for Ura⁺) or without (for Leu⁺) 0.2% w/v casamino acids and 50 mM sodium-potassium phosphate buffer, pH 6.8), supplemented with uracil (440 mg/L) or leucine (440 mg/L) depending on the auxotrophic requirements. The detection of β-glucosidase activity in solid YNBcasa medium was achieved by incorporating 1.0 mM p-nitrophenyl-β-D-glucoside (pNPGlc) (Guo et al., 2011). For β-glucosidase characterization, enzymes were produced in YTD medium (1% w/v yeast extract, 2% w/v tryptone, 5% w/v glucose and 100 mM phosphate buffer, pH 6.8). To compare the efficiency of recombinant β-glucosidase to degrade cellobiose and cellodextrin with respect to cell growth, yeasts were aerobically cultivated in YNBcasa medium, containing 5 g/L cellobiose or cello-oligosaccharides (C3-C6), and defined medium containing vitamins, trace elements (Verduyn et al., 1992) and salts, including 3.5 g/L (NH₄)₂SO₄, 3.0 g/L K₂HPO₄, 3.0 g/L NaH₂PO₄ and 1.0 g/L MgSO₄.7H₂O with 10 g/L cellobiose. For lipid production using cellulose as the carbon source, Y. lipolytica strains were grown in defined media supplemented with 50 g/L Avicel PH-101.

### 1.2. Plasmid Constructions

The plasmids constructed in the present study are summarized in Table 2, and all primers are listed in Table 3 below.

TABLE 2
Plasmids used or created in the present study

| Plasmids | Description | Source of reference |
|---|---|---|
| JMP62UraTEF | URA3, TEF_{P}-XPR_{T} | Haddouche et al., 2011 |
| JMP62LeuTEF | LEU2, TEF_{P}-XPR_{T} | Nicaud et al., 2002 |
| JMP62UraTB1 | URA3, TEF_{P}-BGL1-XPR_{T} | This study |
| JMP62UraTB2 | URA3, TEF_{P}-BGL2-XPR_{T} | This study |
| JMP62LeuTB2 | LEU2, TEF_{P}-BGL2-XPR_{T} | This study |
| JMP62UraTB12 | URA3, TEF_{P}-BGL1-XPR_{T}, | This study |
| | TEF_{P}-BGL2-XPR_{T} | |

TABLE 3
The sequences of the oligonucleotide primers used in this study

| Primer | Sequence (5′-3′) | Restriction | SEQ ID |
|---|---|---|---|
| names | Restriction sites are italic/underlined | sites | NO: |
| FA1 | CG^{a}GGATCCCGCGATGATCTTCTCTCTGCAACTACT | BamHI | 16 |
| | AC | | |
| RB1 | CGCCTAGGCTACAAAGTGAAAGTCTCACATAGC | AvrII | 17 |
| FA2 | CCCAAGCTTGGGTTTGGAGGGGGTGAAAAA | HindIII | 18 |
| RB2 | CCCAAGCTTGGGCTAAAGACCTAACCAATTCTTAG | HindIII | 19 |
| | TCT | | |
| FA3 | CGGGATCCCGCGATGATTGCAAAAATACCCC | BamHI | 20 |
| RB3 | CGCCTAGGCTACTGGAGAGTAAAGGACTCG | AvrII | 21 |
| FA4 | CGGGATCCCGCGATGCTCGCATTCGTCCTAC | BamHI | 22 |
| RB4 | CGGGATCCCGCTACTTGAGAGTGAAGCTGGTG | BamHI | 23 |
| FA5 | CGGGATCCCGCGATGGCTCCACCCCCGCCTCCT | BamHI | 24 |
| RB5 | CGCCTAGGTTAAGCAATCGTGATGCGACCAAGG | AvrII | 25 |
| FA6 | CGCCTAGGCGCGATGGAGGAATTATCGGAGGC | AvrII | 26 |
| RB6 | CGCCTAGGCTACCGGCTGAACTTCTCTTC | AvrII | 27 |
| RB- | bCGCCTAGGTTAATGATGGTGATGATGGTGGCTGCCG | | 28 |
| His1 | CGCGGCACCAGCCTAGGCAAAGTGAAAGTCTCA | | |
| RB- | CCCAAGCTTGGGTTAATGATGGTGATGATGGTGGCTG | | 29 |
| His2 | CCGCGCGGCACCAGCCTAGGAAGACCTAACCAATT | | |
| | CTTA | | |
| RB- | CGCCTAGGTTAATGATGGTGATGATGGTGGCTGCCGC | | 30 |
| His3 | GCGGCACCAGCCTAGGCTGGAGAGTAAAGGA | | |
| RB- | CGGGATCCCGTTAATGATGGTGATGATGGTGGCTGCC | | 31 |
| His4 | GCGCGGCACCAGCCTAGGCTTGAGAGTGAAGCT | | |
| RB- | CGCCTAGGTTAATGATGGTGATGATGGTGGCTGCCGC | | 32 |
| His5 | GCGGCACCAGCCTAGGAGCAATCGTGATGC | | |
| RB- | CGCCTAGGTTAATGATGGTGATGATGGTGGCTGCCGC | | 33 |
| His6 | GCGGCACCAGCCTAGGCTGAACTTCTCTTCC | | |

^{a}restriction site with corresponding restriction enzyme.
^{b}His-tag introduced into the corresponding genes.

Briefly, these vectors contain the Y. lipolytica TEF promoter and either the URA3ex or LEU2ex excisable selection markers, which are flanked by loxP sites and a Zeta fragment that serves as the homologous integration site (Fickers et al., 2003). Regarding β-glucosidases, six putative gene candidates (Sequences YALI0F16027g, YALI0F01672g, YALI0D18381g, YALI0B14289g, YALI0B14333g, YALI0E20185g available at Genome Resources from Yeast Chromosomes: http://gryc.inra.fr/) were identified (See Table 4 below).

TABLE 4
Six putative β-glucosidase coding genes identified by the conserved
glycosyl hydrolase family 3 N and/or 3C terminal domain.

| | | GenBank | | |
|---|---|---|---|---|
| Gene | | Accession | | *Signal |
| Locus_tag | Similar to | numbers | Identities | Peptide |
| YALI0B14289g | Saccharomycopsis fibuligera | AAA34314.1 | 50.42% | Identified |
| | Beta-glucosidase 1 | | | |
| YALI0B14333g | Saccharomycopsis fibuligera | AAA34315.1 | 45.22% | — |
| | Beta-glucosidase 2 | | | |
| YALI0D18381g | Talaromyces emersonii | AAL34084.2 | 27.20% | Identified |
| | Beta-glucosidase | | | |
| YALI0E20185g | Candida albicans | XP_716473.1 | 26.99% | — |
| | Beta-glucosidase | | | |
| YALI0F01672g | Kluyveromyces marxianus | ACY95404.1 | 50.56% | — |
| | Beta-glucosidase | | | |
| YALI0F16027g | Saccharomycopsis fibuligera | AAA34315.1 | 49.5% | Identified |
| | Beta-glucosidase 2 | | | |

*Identification of signal peptide is done by SignalP 4.1

For the expression of wild-type and His6-tagged proteins, the genes were amplified by PCR using FA (1-6) as forward primers and RB (1-6) or RB-His (1-6) as reverse primers, respectively. The PCR fragments were digested using either BamHI/AvrII, or HindIII/AvrII, and inserted into the plasmid JMP62 UraTEF at the corresponding sites.

After construction, all expression vectors were verified by DNA sequencing (GATC Biotech, Konstanz, Germany). For Y. lipolytica transformation, vectors were digested using NotI, thus generating a linear DNA with Zeta sequences at both extremities, and purified. Then the linear DNA fragments were introduced into the Zeta docking platform of Y. lipolytica JMY1212 Zeta, or randomly into the genome of Δpox strain using the lithium acetate method (Duquesne et al., 2012). Transformants were tested for β-glucosidase activity on YNB glucose plate containing pNPGlc and for growth on cellobiose using solid YNB cellobiose plates. Clones displaying both activities were retained for further analysis.

### 1.3. Measurement of Enzyme Activity

β-Glucosidase activity was measured by quantifying the release of pNP (p-nitroplienol) from pNPGlc as described previously (Guo et al., 2011). One unit of pNPGlcase activity was defined as the amount of enzyme required to release 1 μmol pNP per min. Cellobiose phosphorylase activity was assayed by measuring the formation of Glc-1P from cellobiose as described previously (Reichenbecher et al., 1997). One unit of activity (U) was defined as the amount of enzyme required to release 1 μmol Glc-1P per min. All protein concentrations were measured using the Bradford method and bovine serum albumin as a standard (Bradford, 1976).

### 1.4. Western Blot Analysis

Western blotting of proteins was performed as described by Duquesne et al. (2014). Crude supernatant and cell-free extracts of Y. lipolytica JMY1212 expressing putative β-glucosidases fused with the His6 tag were concentrated 10-fold using an ultra-centrifugation filter unit (Amicon® Ultra-4 10 kDa cut-off, Merk Millipore, Bedford, Mass., USA). Blots were sequentially treated with mouse non position-specific His-Tag antibody 1:2500 (THE™ from Genscript, Piscataway, N.J., US) and the alkaline phosphatase-conjugated goat anti-mouse IgG.

### 1.5. Subcellular Fractionation and Enzyme Localization

Fractionation of yeast cells was carried out as described by Cummings and Fowler (1996), with slight modifications. Briefly, yeasts were cultivated until a cellular density of 6×10⁷ cells/mL was reached. Then, to quantify total β-glucosidase activity, a 50 mL sample was taken and subjected to centrifugation at 8,000×g for 5 min at 4° C. thus isolating a cell pellet and supernatant. The cell pellet was disrupted in Tris-HCl buffer (50 mM, pH 7.4, 3 mM EDTA and 0.5 mM PMSF) using a MP FastPrep-24 Instrument (MP Biomedicals Inc.). β-Glucosidase activity in both the cell lysate and the supernatant was determined as described earlier in order to estimate total β-glucosidase activity. Using a second 50 mL yeast culture, a cell pellet containing approximately 2×10⁸ cells/mL was obtained by centrifugation and then treated with zymolyase 100T at 10 mg/mL (Seikagaku corp roger) in 15 mL of sorbitol buffer (1 M sorbitol, 50 mM Tris-HCl, pH 7.4, 2 mM dithiothreitol, 10 mM MgCl₂, 20 mM-sodium azide, 0.5 mM PMSF) at 30° C. with gentle shaking. Protoplast formation was monitored using a microscope until ≧99% of the cells was lysed when. SDS was added (1% SDS w/v). The solid protoplast fraction was then separated from the supernatant by centrifugation (1000 rpm for 5 min at 4° C.) and the latter was designated as the periplasmic fraction. The protoplasts were re-suspended in Tris-HCl buffer (50 mM Tris-HCl, pH 7.4) and disrupted by vortex in the presence of glass beads (0.4-0.45 mm). The homogenate was centrifuged (20,000×g for 2 h at 4° C.) and the supernatant and solid fractions were designated as the cytoplasmic and membrane fraction respectively. Prior to enzyme assays, the membrane fraction was suspended in citrate buffer.

1.6. Purification ofβ-Glucosidases

Y. lipolytica JMY1212 overproducing Bgl1-His6 and Bgl2 were grown in 200 mL YTD medium at 130 rpm, 28° C. for 36 h before centrifugation at 8,000×g for 5 min. For purification of Bgl1-His6, the cell pellet was washed, suspended in 50 mL phosphate buffer (50 mM, pH 7.4) and homogenized over a 3-min period using a MP FastPrep-24 Instrument. After centrifugation (8,000×g for 5 min at 4° C.), the supernatant was applied to 2 mL of TALON Metal Affinity Resin (Clontech Takara-Bio, Kyoto, Japan) and protein was eluted using imidazole buffer according to the manufacturer's instructions.

For purification of Bgl2, the culture supernatant was concentrated 5-fold using an Amicone Ultra-4 Centrifugal Filter Unit with 30 kDa cut-off (Merk Millipore, Bedford, Mass., USA). Thee concentrated sample was then loaded onto a Q Sepharose™ High Performance column (Hiload, 1.6×10 cm, Pharmacia Biotech), equilibrated with Tris-buffer (20 mM, pH 8.0). The column was washed first with equilibration buffer (2 bed volumes) before applying a linear gradient of 0-1.0 M NaCl in Tris-buffer (20 mM, pH7.4) at a flow rate of 1.0 mL/min (Pharmacia Biotech ÄKTA). Eluted fractions were collected and assayed for β-glucosidase activity. All fractions displaying activity were pooled, desalted and concentrated using an Amicon ultra-filtration unit equipped with a PM-10 membrane (Millipore), before being applied to a Superdex 200 column (1.0×30 cm, Pharmacia Biotech) equilibrated in Tris-sodium buffer (20 mM Tris-HCl, 150 mM NaCl, pH 7.4). Protein species were separated at a flow rate of 0.5 mL/min. Fractions were collected and analyzed by SDS-PAGE in order to ascertain purity and estimate the approximate molecular weights of Bgl1-His6 and Bgl2. All fractions satisfying the purity criterion (>95% purity) were pooled and retained for further work.

### 1.6. Deglycosylation and N-Terminal Amino Acid Sequencing

Purified Bgl1-His6 and Bgl2 were treated with endoglycosidase H (New England Biolabs, Beverly, Mass., USA) according to the manufacturer's instructions. After deglycosylation, the protein species displaying Mᵣ (relative molecular mass) closest to those of the theoretical (predicted using Protparam, http://web.expasy.org/protparam/) of Bgl1-His6 and Bgl2 were excised and submitted to N-terminal amino acid sequencing (PISSARO platform, Rouen, France).

### 1.7. Physicochemical Characteristics of β-Glucosidases

Optimal temperatures and pH for the activity of Bgl1-His6 and Bgl2 were determined using pNPGlc as the substrate. Assays were either performed at pH 5.0 and various temperatures (30-70° C.), or at 30° C. in variable pH conditions (2.0 to 8.0) using either 50 mM glycine-HCl (pH 2.0), 50 mM citrate/acetate (pH 3.0-7.2), or potassium phosphate (pH 7.0-8.2) buffer. When the temperature was varied, the pH of the citrate buffer was adjusted accordingly. Stability of Bgl1-His6 and Bgl2 depending on pH and temperature was analysed as follows: enzymes were incubated at 30° C. for up to 2 h at various pH values (2.0 to 8.0), or at various temperatures (30-70° C.) for up to 2 h in 50 mM citrate buffer, pH 5.0. Residual glucosidase activity was then assayed at 30° C. in 50 mM citrate buffer, pH 5.0.

### 1.8. Substrate Specificity and Enzyme Kinetics

The substrate specificity of Bgl1-His6 and Bgl2 was investigated by assaying for activity on the aryl-glycosides pNP-β-D-glucopyranoside, pNP-α-D-glucopyranoside, pNP-β-D-galactopyranoside, pNP-β-D-xylopyranoside and pNP-β-D-cellobioside, and on the oligosaccharides cellobiose, cellotriose, cellotetraose, cellopentaose, cellohexaose, sophorose, laminaribiose, gentiobiose, methylglucoside and octylglucoside. When using aryl-substrates, the standard assay method was employed, simply replacing pNPGlc by another substrate as appropriate. For oligosaccharides, the release of glucose was quantified using an enzyme kit (D-Fructose/D-Glucose Assay Kit, liquid stable, Megazyme). To study the Michaelis-Menten parameters K_{M}, Vₘₐₓ and k_{cat}, Bgl1-His6 (0.120 nM) or Bgl2 (0.13 nM) were added to reaction mixtures containing different substrate concentrations: 0.25-5 mM cellobiose, 0.25-5 mM cellotriose, 0.25-5 mM cellotetraose, 0.25-5 mM cellopentaose, 0.25-5 mM cellohexaose, 0.2-4 mM sophorose, 0.1-2 mM laminaribiose, 0.1-2 mM gentiobiose, 0.5-20 mM methylglucoside and 0.2-4 mM octylglucoside. Initial rates were fitted to the Michaelis-Menten kinetic equation using a nonlinear regression (SigmaPlot 10) to extract the apparent K_{M} and k_{cat} (Segel, 1993).

### 1.9. Yeast Growth and Lipid Production

Yeast growth on cellobiose and cellodextrins was performed in a 40-well microplate. A single colony from a fresh YPD plate was transferred into 5 mL of defined medium containing 10g/L of glucose and pre-cultured until the mid-exponential phase. The cells were then harvested, washed, suspended in sterile water and used to inoculate 200 μL YNBcasa media containing 5 g/L cellobiose or cellodextrins in the microplate, achieving an initial OD₆₀₀ of 0.1. This culture was grown in a microplate reader (Spectrostar Omega, BMG Labtech, Germany) at 30° C. with continuous shaking (150 rpm) and automatic OD₆₀₀ recording.

Similarly, for lipid production a fresh yeast culture in exponential phase was used to inoculate 50 mL defined medium containing 50 g/L Avicel in Erlenmeyer flasks, achieving an initial OD₆₀₀ of 1.0. Celluclast 1.5 L (60 FPU/mL, gift from Novozymes, Denmark) was added (7.5 U/g cellulose) and growth was pursued for 5 days (30° C., 150 rpm). Samples were taken at regular intervals to determine concentrations of biomass, glucose, cellobiose and citric acid. In parallel, two control experiments were conducted under the same conditions, with or without the addition of extra β-glucosidase (810 IU/mL Novozyme 188, gift from Novozyme, Denmark) at 12.0 IU/g cellulose as recommended (Lan et al., 2013).

### 1.10. Analysis of Product Formation and Determination of Dry Cell Weight

To determine the concentration of substrates and extracellular metabolites, three aliquots (1.5 mL each) of cultures were rapidly frozen in liquid nitrogen and then thawed on ice before centrifugation (8,000×g for 5 min at 4° C.) to recover supernatants for analysis. Glucose, cellobiose and citric acid were measured using an Aminex HPX87-H column (Bio-Rad Laboratories, Germany), operating at 50° C. using a mobile phase (5 mM H₂SO₄) flowing at a rate of 0.5 mL/min. Glucose and cellobiose were detected using a Shodex RI-101 refractive index detector (Showa Denko, New York, N.Y.), while citric acid was detected using an UV detector at 210 nm (Dionex, Sunnyvale, Calif.).

To determine the dry cell weight, three aliquots (5 mL each) of cultures were filtered through pre-weighed PES filters (0.45 μm; Sartorius Biolab, Germany). The biomass retained by the filters was washed, dried in a microwave oven at 150 W for 15 min, and then placed in a desiccator before weighing. The biomass yield was calculated as the ratio of the amount of biomass obtained divided by the amount of carbon source consumed.

Lipids were extracted from freeze-dried cells (˜10 mg) and methylated as described previously (Browse et al., 1986). During the lipid extraction, C17:0 (Sigma) (50 μg) was added as the internal standard and fatty acid methyl esters (FAMEs) were analyzed by gas chromatography (6890N Network GC System, Agilent, USA). The measurements were performed in a split mode (1 μL at 250° C.), with helium as the carrier gas (2 mL/min). FAMEs were separated on a HP-5 GC column (30 m×0.32 mm I.D., 0.5-μm film thickness, Agilent, USA). The temperature program was 120° C., ramped to 180° C. (10° C./min) for 6 min, 183° C. (0.33°C./min) for 9 min, and 250° C. (15° C./min) for 5 min. Detection was performed using a flame ionization detector (FID) at 270° C. (2.0 pA). FAMEs were quantified by comparing their profiles with that of standards of known concentration.

### 2. Results

### 2.1. Identification of Genes Encoding Active β-Glucosidases in Y. Lipolytica

Analysis of the Y. lipolytica genome using BLAST revealed the presence of six sequences that were identified as putative family GH3 β-glucosidases (See Table 4 above) on the basis of high amino sequence identity with other yeast β-glucosidases. However, in the absence of biochemical data it was impossible to assert at this stage that these sequences actually encode β-glucosidases, since family GH3 contains glycoside hydrolases that display other specificities and also because Y. lipolytica does not grow on cellobiose and has not been found to express a detectable level of β-glucosidase activity (See FIG. 1). In this respect, it was observed that overexpression of BGL1 (YALI0F16027g) or BGL2 (YALI0B14289g) in Y. lipolytica (strains ZetaB1 and ZetaB2 respectively) conferred the ability to grow on solid medium containing cellobiose as the sole carbon source. Additionally, when these recombinant strains were grown on YNB-pNPGlc plates, yellow halos surrounding the colonies were clearly visualized, indicating β-glucosidase activity (FIG. 1). Finally, after growth in liquid YTD medium, β-glucosidase activity could be measured in the cell extract of ZetaB 1 (3.2±0.2 IU/mg) and in the culture supernatant of ZetaB2 (2.6±0.1 U/mL), while much lower activities were measured in the culture supernatant of ZetaB1 (0.33±0.02 U/mL) and in the cell extract of ZetaB2 (0.42±0.01 IU/mg).

To further investigate the production of β-glucosidases, Y. lipolytica expressing His6-tagged β-glucosidases were constructed and western blot analysis was carried out using anti-His6 antibodies. This revealed that only Bgl1-His6 was detectable in both the culture supernatant and cell extract (FIGS. 2a, b), consistent with the fact that expression of Bgl2-His6 failed to reveal any detectable β-glucosidase activity, although expression of the native BGL2 sequence was successful.

### 2.2. Localization of β-Glucosidases in ZetaB1 and ZetaB2

To determine the localization of Bgl1 and Bgl2, yeast cells expressing these enzymes were fractionated, generating on one hand extracellular samples (culture supernatant), and on the other cell-associated periplasmic, cytoplasmic and membrane fractions. Measurement of the β-glucosidase activities in each of these fractions revealed that Bgl1 was primarily localized in the periplasm, while Bgl2 was mainly in the supernatant (Table 5 below).

TABLE 5
Distribution of β-glucosidase activity
in recombinant strains ZetaB1 and ZetaB2

Relative enzyme activity^{a}

| Fraction | Bgl1 (%) | Bgl2 (%) |
|---|---|---|
| Total | 100 | 100 |
| Growth medium | 2.3 ± 0.4 | 79.6 ± 1.2 |
| Periplasm | 60.7 ± 1.0 | 25.8 ± 1.0 |
| Cytoplasm | 30.0 ± 0.5 | 4.8 ± 0.8 |
| Membrane | 8.1 ± 0.7 | 3.7 ± 0.3 |

^{a}triplicate experiments.
±the standard deviation.

Activity was assayed with pNPGlc.

Accounting for the limits of the experimental methods employed, Bgl1 was also quite present in the cytoplasmic fraction and the presence of Bgl2 in the periplasm was also significant. Overall, these data are consistent with the conclusion that Bgl1 is probably localized in the periplasmic space, while Bgl2 is secreted to the culture medium.

### 2.3. Production, Purification and Characterization of Bgl1 and Bgl2

Production of Bgl1-His6 and native Bgl2 was achieved by growing the appropriate Y. lipolytica strains on YTD in aerobic cultivations, with expression of both enzymes increasing until complete depletion of glucose was reached (36 h).

Regarding purification of Bgl1-His6, yeast cells arising from a 200-mL culture volume yielded approximately 550 U (170 mg) of enzyme in the crude cell extract. However, after purification only 17% of Bgl1-His6 was recovered (Table 6 below).

TABLE 6
Purification of intracellular Bgl1-His6 and extracellular
Bgl2 produced by Y. lipolytica overexpressing strains.

| | | | | | | Yield |
|---|---|---|---|---|---|---|
| | | Total | Total | Specific | Fold | (%) |
| Enzyme and purification | | protein | activity | activity | purifi- | Recov- |
| method | | (mg) | (U) | (U/mg) | cation | ery |
| Bgl1- | Filtrate | 169.7 | 543.0 | 3.2 | — | 100 |
| His6 | TALON His-tag^{a} | 0.9 | 92.5 | 102.8 | 32.1 | 17.0 |
| Bgl2 | Culture filtrate | 2302.5 | 530.2 | 0.23 | — | 100 |
| | Ultra filtration | 1986.4 | 510.5 | 0.26 | 1.1 | 96.3 |
| | Ion exchange | 235.3 | 478.5 | 2.0 | 7.7 | 90.2 |
| | Gel filtration | 1.8 | 46.4 | 25.8 | 112.2 | 8.8 |

^{a}Specific activity was tested on pNPGlc

In the case of Bgl2, a two-step protocol using anion exchange chromatography and gel filtration allowed its purification to near homogeneity, but led to significant loss of protein (8.8% recovery). SDS-PAGE analysis of the two purified protein samples revealed that the Mᵣ of Bgl1-His6 was slightly higher than expected (theoretical Mᵣ=92.1 kDa) (FIG. 2c), while that of Bgl2 was significantly higher (>250 kDa) than the expected Mᵣ of 94.6 kDa with presence of the potential signal peptide (FIG. 2d). To understand this anomaly, the amino acid sequence of Bgl2 was analyzed using the glycosylation predictor GlycoEP (http://www.imtech.res.in/raghava/glycoep/; Chauhan et al., 2013). This revealed that Bgl2 harbors 18 potential N-glycosylation sites. Therefore, to investigate the actual glycosylation state of recombinant Bgl2, the purified protein was deglycosylated using endoglycosidase H treatment. After deglycosylation and SDS-PAGE analysis, the Mᵣ of the recombinant Bgl2 was estimated to be approximately 95 kDa, consistent with the theoretical Mᵣ (FIG. 2d). Finally, N-terminal amino acid sequence analysis of Bgl1 and Bgl2 confirmed the identity of the two proteins and revealed the signal peptide of the two Bgls (SECS ID NO: 4 and 6 respectively).

Preliminary characterization of Bgl1-His6 and Bgl2 using pNPGlc as the substrate revealed that Bgl1 was 5-fold more active (102.8 U/mg) on this substrate than Bgl2 (25.8 U/mg). The activity of Bgl1-His6 was highest at approximately pH 4.5 and 45° C., and was stable in the pH range of 4.0-5.0 and below 40° C. Regarding Bgl2, it was found to display highest activity at pH 4.0 and 50° C., and was stable in the pH range of 3.5-7.0 and below 50° C. (FIGS. 3 and 4). It is noteworthy that deglycosylation of Bgl2 led to a 60% decrease in specific activity, which was probably due to its instability at 40° C. (FIG. 5).

### 2.4. Substrate Specificity and Kinetic Parameters of Bgl1 and Bgl2

The substrate specificity of the purified β-glucosidases was examined using different substrates displaying α and β configurations. The results showed that both β-glucosidases were maximally active against pNPGlc (FIG. 6). However, using activity on pNPGlc as the benchmark, it is noteworthy that both enzymes were active on pNP-β-D-cellobioside (Bgl1-His6, 24% and Bgl2, 27%), but only Bgl1-His6 displayed significant activity (10%) on pNP-β-D-xylopyranoside. Neither enzyme displayed activity on pNP-β-D-galactopyranoside and pNP-α-D-glucopyranoside.

When the activity of Bgl1-His6 and Bgl2 on cellobiose was compared with that on other oligosaccharides, it was found that both enzymes displayed highest activity on laminaribiose (β-1, 3-linkage), followed by gentiobiose (β-1, 6-linkage), octylglucoside, sophorose (β-1, 2-linkage), cello-oligosaccharides (C3-C6) and cellobiose (β-1, 4-linkage). It is noteworthy that the hydrolytic activity of Bgl1-His6 was less dependent on the chain length of cello-oligosaccharides, while hydrolytic activity of Bgl2 increased as the length of cello-oligosaccharides increased. Both enzymes recognized methylglucoside as substrate, but the hydrolytic activities were low compared with the other substrates (FIG. 6), indicating that correct occupation of subsite +1 is important for catalysis.

The determination of the apparent kinetic parameters of reactions catalyzed by Bgl1-His6 and 2 and containing various glucosyl disaccharides and cello-oligosaccharides revealed that the values of K_{M}(app) and k_{cat}/K_{M} for Bgl2-catalyzed reactions increased as a function, of degree of polymerization (DP) of the cello-oligosaccharides (see Table 7 below).

TABLE 7
Kinetic parameters of Y. lipolytica Bgls for various glycoside-substrates^{a}

| | | Bgl1-His6 | | | Bgl2 | | |
|---|---|---|---|---|---|---|---|
| | | K_{M} | k_{cat} | k_{cat}/K_{M} | K_{M} | k_{cat} | k_{cat}/K_{M} |
| Substrate | Linkage | (mM) | (s⁻¹) | (mM⁻¹s⁻¹) | (mM) | (s⁻¹) | (mM⁻¹s⁻¹) |
| Cellobiose | Glc × 2, β-1,4 | 0.26 | 21.1 | 81.1 | 0.79 | 5.1 | 6.5 |
| Cellotriose | Glc × 3, β-1,4 | 0.43 | 20.5 | 47.7 | 0.99 | 9.5 | 9.6 |
| Cellotetraose | Glc × 4, β-1,4 | 1.89 | 30.9 | 16.3 | 1.86 | 20.6 | 11 |
| Cellopentaose | Glc × 5, β-1,4 | 2.18 | 29.5 | 13.5 | 2.24 | 27.5 | 12.3 |
| Cellohexaose | Glc × 6, β-1,4 | 3.01 | 31.5 | 10.5 | 2.37 | 30.5 | 12.9 |
| Sophorose | Glc × 2, β-1,2 | 2.25 | 28.4 | 14.8 | 2.4 | 41.2 | 17.2 |
| Laminaribiose | Glc × 2, β-1,3 | 0.68 | 75.6 | 110.7 | 0.89 | 211.1 | 237.2 |
| Gentiobiose | Glc × 2, β-1,6 | 1.16 | 43.6 | 37.6 | 1.84 | 186.5 | 101.4 |
| Methylglucoside | C = 1 | 15 | 15 | 1 | 6.23 | 34.1 | 5.5 |
| Octylglucosidc | C = 8 | 0.86 | 32.8 | 38.1 | 1.3 | 111.1 | 85.2 |

^{a}The mean values of three independent experiments are shown and the standard deviation is below 10%. Hydrolytic activities for the substrate were determined from the amount of released glucose and the kinetic parameters were calculated as described in Materials and Method.

In the case of Bgl1-His6, increased DP was associated with increased K_{M}(app) values, but not k_{cat}/K_{M} values. Overall, considering the performance constant (k_{cat}/K_{M}), cellobiose and cellohexaose were the best substrates for Bgl1-His6 and Bgl2 respectively. Additionally, the performance constant of Bgl1-His6 measured on cellobiose was 12.5-fold higher than that describing Bgl2. Regarding other glucosyl substrates (i.e. those containing linkages other than β-1,4), both Bgls displayed the highest performance constants on laminaribiose. Nevertheless, comparison of the performance constants on each of the substrates revealed that Bgl2 is less regioselective, since the k_{cat}/K_{M} values were always lower in reactions catalyzed by Bgl1-His6 (86% for sophorose, 47% for laminaribiose, 37% for gentiobiose, 18% for methylglucoside and 45% for octylglucoside). Finally, the lowest performance constants for both Bgls were measured for reactions containing methylglucoside.

2.5. Cellobiose and Cello-Oligosaccharide Fermentation with Y. Lipolvtica Recombinant Strains

Yeast strains ZetaB1 expressing BGL1 and ZetaB2 expressing BGL2 were grown in micro cultivation plates under aerobic conditions in the presence of cellobiose or cellodextrins as sole carbon sources, using wild type Y. lipolytica ZetaW as the control. The maximum specific growth rates (μₘₐₓ) of the transformants on cellobiose were essentially the same as that of the control grown on glucose (FIGS. 7a, b). ZetaB 1 grew faster than ZetaB2 on cellobiose and cellodextrins (FIGS. 7b-f), while the control was unable to grow on either of these substrates.

Further characterization of the recombinant strains in shake flask cultures showed that ZetaB1 consumed 8 g/L cellobiose over 48 h. However, upon further incubation, the remaining cellobiose (2 g/L) was not consumed (FIG. 8). In contrast, ZetaB2 consumed all of the cellobiose (10 g/L) over 64 and 72 h respectively (FIG. 8). Furthermore, ZetaB1 sustained a specific aerobic growth rate (μₘₐₓ) of 0.16 h⁻¹ (identical to that on glucose), whereas ZetaB2 exhibited a long lag phase on cellobiose after which two subsequent growth phases (μₘₐₓ values of 0.08 h⁻¹ and then 0.16 h⁻¹) were observed (FIG. 8 and Table 8 below).

TABLE 8
Comparison of growth and biomass yield of Y. lipolytica JMY1212
control and recombinant strains in aerobic cellobiose cultivation

| Parameter | Control | ZetaB1 | ZetaB2 | ZetaB12 |
|---|---|---|---|---|
| μₘₐₓ (h⁻¹) on glucose | 0.15 ± 0.01 | 0.16 ± 0.01 | 0.16 ± 0.01 | 0.16 ± 0.01 |
| μₘₐₓ (h⁻¹) on cellobiose | N.A. | 0.16 ± 0.01 | 0.15 ± 0.01 | 0.16 ± 0.01 |
| Y_{X/S} (DCW-g/g cello) | N.A. | 0.52 ± 0.01 | 0.53 ± 0.01 | 0.50 ± 0.01 |
| Residue cellobiose 60 h (%) | N.A. | 17.2 ± 1.0 | 7.2 ± 0.1 | 1.0 ± 0.3 |

±the standard deviation.
N.A. = Not available

In order to combine the advantages procured by the overexpression of BGL1 and BGL2 (i.e. faster growth rate and higher cellobiose utilization respectively), the two BGL sequences were cloned into JMY1212, thus yielding ZetaB12. During cultivation on cellobiose, the performance of ZetaB12 was the best among all the recombinant strains. It showed similar growth rate to that of ZetaB1 and consumed 10 g/L of cellobiose within 40 h.

### 2.6. Characterization of Cellulose-Based Lipid Production by Recombinant Y. Lipolytica Strains

A strategy to increase lipid accumulation was based on the disruption of the β-oxidative metabolism, through the deletion of the 6 POX genes (POX1 to POX6) that encode the peroxisomal acyl-coenzyme oxidases as has been done in Δpox strain (Beopoulos et al., 2008). To investigate whether the recombinant Y. lipolytica strains could be useful in a consolidated bioprocess for lipid production, recombinant Y. lipolytica ΔpoxB1, ΔpoxB2, ΔpoxB12 and ΔpoxPT were grown, on cellulose in the presence of Celluclast 1.5 L. Even though this cocktail is reputedly β-glucosidase-deficient, to avoid any problems (i.e. spurious results linked to the presence of β-glucosidase in Celluclast) the Celluclast loading was kept low (7.5 FPU/g cellulose), and control experiments containing the prototrophic Y. lipolytica ΔpoxW strain grown in the presence of Celluclast 1.5 L with or without β-glucosidase supplementation were performed. During the initial 6 h of cultivation an accumulation of reducing sugars was observed in all of the cultures, which was attributed to Celluclast 1.5 L-mediated cellulose hydrolysis. However, further monitoring revealed that after 12-h growth, less reducing sugars were present in the Y. lipolytica ΔpoxB12 culture (2.7 g/L) compared to the other cultures (FIG. 9b). Moreover, this observation was correlated with continued yeast growth, whereas the growth of the other cultures stagnated over the same period (FIG. 9b). After 60 h of cultivation, the growth of the ΔpoxB12 reached a stationary phase. At this point the amount of FAMEs had reached 0.8 g/L (FIG. 9c), but further growth did not result in an increase in cellular lipid content, reflecting a limitation of the available energy source. Besides the longer lag phase, the growth of ΔpoxB1 and ΔpoxB2 was similar to that of the control culture supplemented with β-glucosidase. Regarding the control culture, in the absence of β-glucosidase supplementation, growth ceased after 60 h and the cell density of the culture was approximately half that of the other cultures. Moreover, continuous addition of cellulases to the control culture did not procure any obvious increase in growth. When the control was supplemented with β-glucosidase, the amount of cellulose that remained unconsumed (25 g/L) was similar to that of cultures of the Δpox strain expressing β-glucosidases after 5 days of growth and was less than that of the cultures with ΔpoxPT (30 g/L).

### EXAMPLE 2

### Lipid Production on Cellobiose by Y. Lipolytica Over-Expressing BGL1 and BGL2

### 1. Materials and Methods

### 1.1. Strains and Media

The Y. lipolytica strain JMY4086 (Rakicka, et al., 2015), in which the six POX genes (encoding acyl-coenzyme A oxidases) and the TGL4 gene (encoding an intracellular triglyceride lipase) were deleted, and DGA2 (encoding acyl-CoA:diacylglycerol acyltransferase) and GPD1 (encoding glycerol-3 -phosphate dehydrogenase) were overexpressed, was used in this study for lipid overproduction. Minimal medium contains 0.17% w/v yeast nitrogen base YNB, 6% cellobiose w/v (≈C:N ratio at 60), 0.15% w/v NH₄Cl and 50 mM phosphate buffer (pH 6.8) was used for lipid production.

### 1.2. Strain Construction

The LEU2 and URA3, encoding beta-isopropylmalate dehydrogenase and orotidine-5′-phosphate decarboxylase, respectively, were removed from Y. lipolytica JMY4086 as previously described (Fickers et at., 2003). Next, the vectors JMP62LeuTEF-BGL1 and JMP62UraTEF-BGL2 containing expression cassettes encoding YALI_BGL1 (SEQ ID NO: 4) and YALI_BGL2 (SEQ ID NO: 6) respectively, were digested using NotI and then introduced randomly into the genome of Y. lipolytica JMY4086 using the lithium acetate method. Transformants were tested for β-glucosidase activity on YNB glucose plate containing 1.0 mM p-nitrophenyl-β-D-glucoside pNP-Glc (Guo et al., 2011), and for growth on minimal medium containing 5 g/L cellobiose. Clones displaying both activities were retained for further analysis by PCR. The resultant transformant was designated as Y. lipolytica LP-B12.

### 1.3. Lipid Production

For lipid production a fresh yeast culture in exponential phase was used to inoculate 200 mL of defined medium containing 60 g/L cellobiose in Erlenmeyer flasks, achieving an initial OD₆₀₀ of 1.0. Y. lipolytica ΔpoxB12 was used as the control. The growth was pursued for 6 days (30° C., 150 rpm). Samples were taken at regular intervals to determine concentrations of biomass, cellobiose and lipid.

### 1.4. Microscopic Analysis

To visualize lipid bodies, BodiPy® Lipid Probe (2.5 mg/ml in ethanol; Invitrogen) was added to a cell suspension (A_{600 nm} of 5), which was incubated for 10 min at room temperature before the acquisition of images. For this, a Zeiss Axio Imager M2 microscope (Zeiss, Le Pecq, France) equipped with a 100× objective and Zeiss filters 45 and 46 for fluorescent microscopy was employed (excitation/emission maxima ˜503/512 nm). Axiovision 4.8 software (Zeiss, Le Pecq, France) was used for image acquisition.

### 2. Results

### 2.1. Lipid Production on Cellobiose by Y. Lipolytica Overexpressing BGL1 and BGL2

Previous work has shown that Y. lipolytica JMY4086 can accumulate lipids using substrates such as crude glycerol and molasses (Rakicka et al., 2015). To investigate whether the BGLs described in this study can confer cellobiose utilization to this strain, and thus allow it to produce lipids from cellobiose as a carbon source, an engineered Y. lipolytica JMY4086 strain overexpressing BGL1 and BGL2 (LP-B12) was constructed. Using this strain a lipid production experiment was performed in shaker flasks containing 200 mL minimal medium and 60 g/L cellobiose. An appropriate control culture was also included that deployed BGL-producing Y. lipolytica that does not display the lipid producing phenotype (ΔpoxB12). The C/N ratio of the media (60:1) was used in order to create nitrogen limiting conditions necessary for lipid production (Rakicka et al., 2015).

Both the lipid-producing and control strains displayed rapid consumption of cellobiose and microbial biomass accumulation over the first two days (FIG. 10). Moreover, after 2-days the use of a FAME analysis method revealed the low level cellular accumulation of lipids, which represented a little less than 10% of dry cell weight (DCW). Subsequently, the cellular lipid content of Y. lipolytica LP-B12 progressively increased, reaching 35% DCW after 6 days. At this time point, cellobiose was almost depleted (FIGS. 10b, 10c; FIG. 11). In addition to lipid accumulation, biomass accumulation was also pursued over the 6-day period with the final biomass yield being 17.6 g DCW/L (FIG. 10a). In comparison, ΔpoxB12 did not grow after 2 days and the final biomass yield was approximately 13 g-DCW/L. Moreover, monitoring of cellobiose consumption revealed that the control culture failed to use all of the carbon source, since the cellobiose after 4 days of growth was 30 g/L. Accordingly, the maximum cellular lipid concentration reached (after 4 days) by the control culture was 15.3% DCW (FIGS. 10 and 11).

In conclusion, the expression of BGLs in Y. lipolytica is sufficient to confer cellobiose utilization phenotype to the yeast and when this phenotype is combined with that of lipid production, lipid accumulation using cellobiose as the sole carbon source is observed.

### REFERENCES

André, A., et al., 2009. Eng. in Life Sci. 9:468-478.
Belancic, A., et al., 2003. J. Agric. Food Chem. 51:1453-1459.
Beopoulos, A., et al., 2008. Appl. Environ. Microbiol. 74:7779-7789.
Blazeck, J., 2011. Appl. Environ Microbiol. 77: 7905-7914.
Blazeck, J., 2013. Appl. Microbiol Biotechnol. 97:3037-3052.
Blazeck, J., et al., 2014. Nat. Commun. 5:3131.
Bordes, F., et al., 2007. J. Microbiol. Methods. 70:493-502.
Bradford, M. M., 1976. Anal. Biochem. 72:248-254.
Browse, J., et al.,1986, Anal. Biochem. 152:141-145.
Chauhan, J. S., et al., 2013. PLoS ONE. 8:e67008.
Cummings, C., Fowler, T., 1996. Curr. Genet. 29:227-233.
Daroit, D. J., et al., 2008. J. Microbiol. Biotechnol. 18:933-941.
Demirbas, A., 2003. Energy Conyers Manage. 44:2093-2109.
Duff, S. J. B., et al., 1985. Biotechnol. Lett. 7:185-190.
Duquesne, S., et a, 2012. Methods Mol Biol. 861:301-312.
Easterling, E. R., et al., 2009. Bioresour. Technol. 100:356-361.
Fabre, E., et al., 1991. J Biol Chem., 266:3782-3790.
Fakas, S., et al., Appl Microbiol Biotechnol., 2006, 73:676-683.
Fickers, P., et al., 2003. J. Microbiol. Methods. 55:727-737.
Frank, K. and Sippl M. J., 2008. Bioinformatics. 24:2172-2176.
Gaillardin, C., et al., 1987, Curr Genet., 11:369-375.
Galas, E., Romanowska, I., 1996. Appl. Environ. Microbiol. 62:3165-3170.
Gasmi, N., 2011. Appl Microbiol Biotechnol. 89:109-119.
Groenewald, M., et al., 2014. Crit. Rev. Mierobiol. 40:187-206.
González-Pombo, P., et al., 2008. Biotechnol. Lett. 30:1469-1475.
Guo, X., et al, J. Appl. Microbiol., 2000, 89:107-115.
Guo, Z. P, et al., 2011. Enzyme Microb. Technol. 49:105-112.
Gysler, C., et al., 1990, Biotechn Techn., 4:285-290.
Haddouche, R., et al., 2011. Appl. Microbiol. Biotechnol. 91: 1327-1340.
Hashimoto, Y., 1998. Protein Engineering. 11:75-77.
Hedfalk, K. (2012) ‘Codon Optimisation for Heterologous Gene Expression in Yeast’. In Springer Protocols: Methods in Molecular Biology. Recombinant protein production in yeast: methods and protocols. Volume 866 pp. 47-55. Springer Eds.
Ito, H., et al., 1983, J. Bacteriol., 153:163-168.
Klebe, R. J., et al., 1983, Gene, 25:333-341.
Koganesawa, N., et al., 2001, Protein Eng. 14:705-710.
La Grange, D. C., et al, 2010. Appl. Microbiol. Biotechnol. 87:1195-1208.
Lane, S, et al., 2014. Biotechnol Bioeng. In press.
Lazar Z., et al, 2014, Metab. Eng. 2689-99.
Le Dali, M. T., et al., 1994, Curr Genet., 26:38-44.
Leclerc, M., et al., 1987. Appl. Biochem. 9:410-422.
Lee, W. H., et al, 2013. J. Biotechnol. 167:316-322.
Lian, J., et al., 2014. Biotechnol. Bioeng. 111:1521-1531.
Lynd, L. R., et al., 2005. Curr. Opin. Biotechnol. 16:577-583.
Machida, M., et al, 1988. Appl. Environ. Microbiol. 54:3147-3155.
Madzak C., et al., 2000, J Mol Microbiol Biotechnol. 2:207-216.
Madzak, C., et al., 2004, J Biotechnol. 109:63-81.
Madzak, C. and Beckerich, J. M., 2013, Heterologous protein expression and secretion in Yarrowia lipolytica. In: Yarrowia lipolytica: Biotechnological Applications, Microbiology Monographs Vol. 25. Ed: Barth G, Springer, Heidelberg, Germany. pp. 1-76.
Michely, S., et al., 2013. PLoS One. 8:e63356.
Muller, S., et al., 1998, Yeast, 14:1267-1283.
Nan, H., et al., 2014. Appl. Microbiol. Biotechnol. 98:5757-5764.
Nicaud, J-M., et al., 2002. FEMS Yeast Res. 2:371-379.
Orr-Weaver, T. L., et al., 1981, Proc. Natl. Acad. Sci. USA, 78:6354-6358.
Papanikolaou, S., et al., Antoine van Leeuwenhoek, 2001, 80:215-224.
Papanikolaou, S., et al., J. Appl. Microbiol., 2002, 92:737-744.
Papanikolaou, S., et al., Bioresour. Technol., 2008, 99:2419-2428.
Papanikolaou, S., Aggelis, G., 2010. Eur. J. Lipid Sci. Techol. 112:639-654.
Parrou, J. L., François, J., 1997. Anal. Biochem. 1:186-188.
Pedersen, M., Meyer, A. S., 2010. N Biotechnol. 27:739-750.
Petersen, T. N., et al., 2011. N. Methods. 8:785-786.
Pignède, G., et al., 2000. J. Bacteriol. 182:2802-2810.
Rakicka M., et al., 2015. Biotechnol Biofuels. 8:104.
Ratledge C. (1994). Yeasts, moulds, algae and bacteria as sources of lipids. Technological advances in improved and alternative sources of lipids. B. S. Darnel, Kakuda, Y. London, Blackie academic and professional, 235-291.
Ratledge, C., 2005. Single cell oils for the 21th century, in: Cohen, R. (Eds.), Single cell oils. AOCS Press, Champaign, pp. 1-20.
Reichenbecher, M., et al., 1997. Eur. J. Biochem. 247:262-267.
Runguphan, W., Keasling, J. D., 2014. Metab. Eng. 21:103-113.
Shi, S., et al, 2012. Biotechnol. Biofuels. 5:7-16.
Sreekrishna, K., 1997. Gene. 190:55-62.
Stockton, D. C., et al., 1991. Biotechnol. Lett. 13:57-62.
Steen, E. J, et al., 2010: Nature. 463:559-562.
Sun, Y., Cheng, J., 2002. Bioresour. Technol. 83:1-11.
Tatusova, T. A. and Madden, T. L., 1999. FEMS Microbiol. Lett. 174:247-250.
Thevenieau, F., Nicaud, J-M., 2013. OCL. 20:1-8.
Tomme, P., et al., 1995. Adv. Microb. Physiol. 37:1-81.
Valle-Rodriguez, et al., 2014. Appl. Energ. 115:226-232.
Verduyn, C., et al., 1992. Yeast. 8:501-517.
von Heijne, G., 1985. J Mol Biol. 184: 99-105.
Wei, H., et al., 2014, Biotechnology for Biofuels, 7:148.
Wilson, D. B., 2009. Curr. Opin. Biotechnol. 20:295-299.
Yan, T. R., Lin, C. L, 1997. Biosci. Biotechnol. Biochem. 61:965-970.
Zhang, F., et al., 2012. Nat. Biotechnol. 30:354-359.

1. A method for obtaining an oleaginous yeast strain capable of growing on cellobiose as carbon source, wherein said method comprises overexpressing in said strain a β-glucosidase having at least 80% identity with the polypeptide of sequence SEQ ID NO: 1 further comprising a N-terminal signal peptide and a β-glucosidase having at least 80% identity with the polypeptide of sequence SEQ ID NO: 2 further comprising a N-terminal signal peptide.

2. The method of claim 1, wherein the β-glucosidase having at least 80% identity with the polypeptide of sequence SEQ ID NO: 1 and/or the β-glucosidase having at least 80% identity with the polypeptide of sequence SEQ ID NO: 2 are from a Yarrowia strain.

3. The method of claim 2, wherein the β-glucosidase having at least 80% identity with the polypeptide of sequence SEQ ID NO: 1 is selected from the group consisting of SEQ ID NO: 1 and SEQ ID NO: 8.

4. The method of claim 1, wherein the β-glucosidase having at least 80% identity with the polypeptide of sequence SEQ ID NO: 2 has the amino acid sequence SEQ ID NO: 9.

5. The method of claim 1, wherein the β-glucosidase having at least 80% identity with the polypeptide of sequence SEQ ID NO: 2 is selected from the group consisting of SEQ ID NO: 2, SEQ ID NO: 11, SEQ ID NO: 13 and SEQ ID NO: 15.

6. The method of claim 1, wherein the signal peptide of the β-glucosidase having at least 80% identity with the polypeptide of sequence SEQ ID NO: 1 and the signal peptide of the β-glucosidase having at least 80% identity with the polypeptide of sequence SEQ ID NO: 2 are identical or different and are selected from the group consisting of SEQ ID NO: 34 to 39.

7. The method of claim 1, wherein the oleaginous yeast strain is selected from the group consisting of the genus Candida, Cryptoccocus, Lipomyces, Rhodosporidium, Rhodotorula, Trichosporon and Yarrowia.

8. The method of claim 7, wherein the oleaginous yeast strain is a Yarrowia strain.

9. The method of claim 1, wherein the expression or activity of the endogenous isoforms of acyl-coenzymeA oxidases in said oleaginous yeast strain is inhibited.

10. The method of claim 9, wherein said oleaginous yeast strain is a Yarrowia strain and wherein in said strain at least one protein selected from the group consisting of an acyl-CoA:diacylglycerol acyltransferase 2, an acyl-CoA:diacylglycerol acyltransferase 1, a glycerol-3-phosphate dehydrogenase NAD+, an acetyl-CoA carboxylase and a hexokinase is further overexpressed, and/or the expression or activity of at least one endogenous protein selected from the group consisting of the glycerol 3-phosphate dehydrogenase, the triglyceride lipase and the peroxin 10 is further inhibited.

11. The method claim 1, wherein it comprises transforming an oleaginous yeast cell with a recombinant DNA construct for expressing both the β-glucosidases, or with two recombinant DNA constructs for expressing both the β-glucosidases respectively.

12. A mutant oleaginous yeast strain, wherein a β-glucosidase having at least 80% identity with the polypeptide of sequence SEQ ID NO: 1 and a β-glucosidase having at least 80% identity with the polypeptide of sequence SEQ ID NO: 2 are overexpressed and wherein it is obtainable by the method of claim 1.

13. Use of a mutant oleaginous yeast strain as defined in claim 12 for producing lipids from a lignocellulosic biomass.

14. A method of producing lipids, comprising a step of growing a mutant oleaginous yeast strain as defined in claim 12 on a lignocellulosic biomass.

15. An isolated β-glucosidase having an amino acid sequence selected from the group consisting of SEQ ID NO: 7, 8 and 10 to 15.

16. Use of an isolated β-glucosidase of claim 15 for degrading cellobiose.
